(19) United States
(12) Patent Application Publication
(10) Pub. No.: US 2024/0300971 A1
(43) Pub. Date: Sep. 12, 2024

(54) PREPARATION METHOD OF 2,6-DIOXASPIRO [4,5] DECANE DERIVATIVES AND SALTS THEREOF

(71) Applicant: SHANDONG LUYE PHARMACEUTICAL CO., LTD., Yantai (CN)

(72) Inventors: Shuai GAO, Yantai (CN); Yanhua WU, Yantai (CN); Jiye ZHAO, Yantai (CN); Guanghui WANG, Yantai (CN); Wensheng LIAO, Yantai (CN)

(21) Appl. No.: 18/582,382

(22) Filed: Feb. 20, 2024

(30) Foreign Application Priority Data
Feb. 20, 2023 (CN) 202310135610.1

(51) Int. Cl. C07D 493/10

(52) U.S. Cl. CPC C07D 493/10

(57) ABSTRACT

The present disclosure provides a preparation method of 2,6-dioxospiral [4,5] decane derivatives and their salts, which has mild reaction conditions, high reaction conversion rate, simple post-processing, and high product purity and is suitable for industrial production.

### TECHNICAL FIELD

The present invention relates to preparation methods of 2,6-dioxaspiro [4,5] decane derivatives or salts thereof.

### BACKGROUND ART

Opioid receptors are a class of G protein-coupled receptors with opioid peptides acting as ligands, and μ, κ, and δ receptors are the classic three types of opioid receptors. Opioid receptors are widely distributed in the body, but unevenly distributed in the nervous system. Opioid receptors are distributed at higher density in the brain, medial thalamus, ventricles, and periaqueductal gray, and these structures are related to the integration and perception of pain. Opioids are currently the most effective analgesics in clinical practice, but they are often likely to cause some target-related side effects, such as respiratory depression, constipation, etc. The binding of opioid GPCR receptors to ligands can simultaneously affect multiple downstream signaling pathways, including Gi protein signaling pathway and β-Arrestin signaling pathway. Current researches show that the analgesic effect of opioids is derived from the Gi protein signaling pathway of receptors, and the related side effects, such as respiratory depression, constipation, etc., are associated with the β-Arrestin signaling pathway downstream of receptors. An enhanced analgesic effect, a prolonged duration of efficacy, and reduced related adverse reactions are observed in β-Arrestin-2-knockout mice injected with morphine, compared with wild-type mice. Therefore, the Gi protein-biased μ-receptor agonists can selectively activate the Gi signaling pathway and have no or little effect on the β-Arrestin pathway. Hence, the Gi protein-biased μ-receptor agonists can be expected to have a better analgesic effect in clinical use and show a great reduction in opioid-related adverse reactions.

Olinvo® Injection, a Gi protein-biased μ-receptor agonist developed by Travena, was approved by the FDA on Aug. 7, 2020 for the treatment of moderate and severe acute pain.

CN111148515B discloses a series of Gi protein-biased μ-receptor agonists, of which the 2,6-dioxaspiro [4, 5] decane compounds shown below have better analgesic effects.

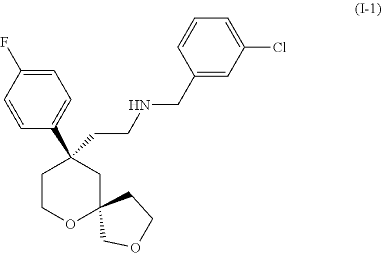 [SMILES: Fc1ccc([C@]2(CCNCc3cccc(Cl)c3)CCO[C@]3(CCOC3)C2)cc1]

The preparation method of the formula (I-1) compound disclosed in CN111148515B requires the use of SFC to separate racemes to obtain a single target isomer. Using Raney nickel as a catalyst for hydrogenation reduction of cyano groups to amino groups, multiple steps were purified using column chromatography. Since Raney Nickel is easy to ignite spontaneously and high-pressure reaction equipment is required for the catalytic hydrogenation reaction, SFC resolution requires high equipment safety; column chromatography is time-consuming and requires the consumption of a large number of solvents, the chromatography solution concentrates and consumes energy and produces a large amount of waste liquids, limiting batch amplification; and the obtained intermediates are oily and difficult to accurately measure; therefore, the disclosed preparation method of CN111148515B cannot be produced industrially on a large scale, and it is necessary to improve it.

### SUMMARY OF THE INVENTION

The invention provides a preparation method of 2,6-dioxaspiro [4,5] decane derivatives and their salts with mild reaction conditions, high conversion rate, simple post-processing, and high product purity, which is suitable for industrial production.

In one aspect of the present invention, a method for preparing compound (I) or a stereoisomer, mixture of stereoisomers, or salts is provided, wherein the method comprises:

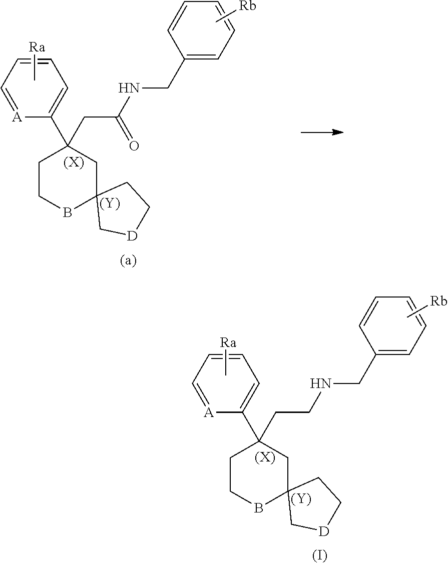

wherein, in the compound (I) and compound (a),
A is selected from C or N;
B and D are independently selected from C, N, or O;
Ra and Rb are independently selected from hydrogen, fluorine, chlorine, bromine, or iodine atoms; and
the carbon atoms with (X) and (Y) are chiral carbon atoms.

In some embodiments, in compound (I) and compound (a), A is N; B and D are both O.

In some embodiments, in compound (I) and compound (a), A is N; B and D are both O, Ra is fluorine atom; and Rb is chlorine atom.

In some embodiments, in compound (I) and compound (a), the configurations of (X) and (Y) in compound (I) and compound (a) are the same or different.

In some embodiments, in compound (I) and compound (a), (X) is S configuration, (Y) is R configuration; or (X) is R configuration, (Y) is S configuration.

In some embodiments, the salts of compound (I) are selected from hydrochloride, hydrobromide, carbonate, phosphate, sulfate, maleate, malonate, benzoate, succinate, octanoate, fumarate, lactate, mandelate, phthalate, besylate, p-toluenesulfonate, citrate, tartrate, and methanesulfonate; preferably maleate.

In some embodiments, compound (I) is selected from:

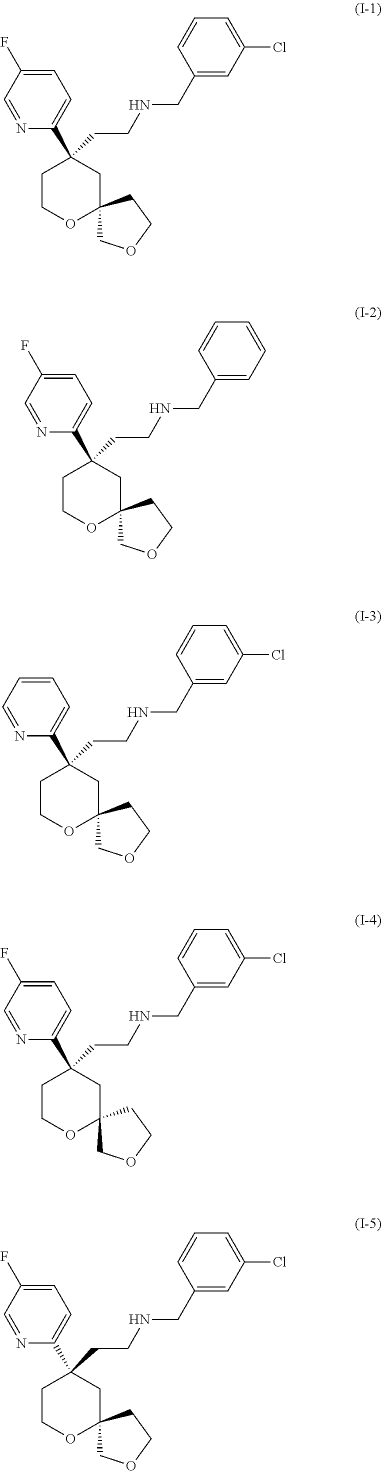 [SMILES: Fc1ccc([C@]2(CCNCc3cccc(Cl)c3)CCO[C@]3(CCOC3)C2)nc1 ; Fc1ccc([C@]2(CCNCc3ccccc3)CCO[C@]3(CCOC3)C2)nc1 ; Clc1cccc(CNCC[C@@]2(c3ccccn3)CCO[C@]3(CCOC3)C2)c1 ; Fc1ccc([C@]2(CCNCc3cccc(Cl)c3)CCO[C@@]3(CCOC3)C2)nc1 ; Fc1ccc([C@@]2(CCNCc3cccc(Cl)c3)CCO[C@]3(CCOC3)C2)nc1]

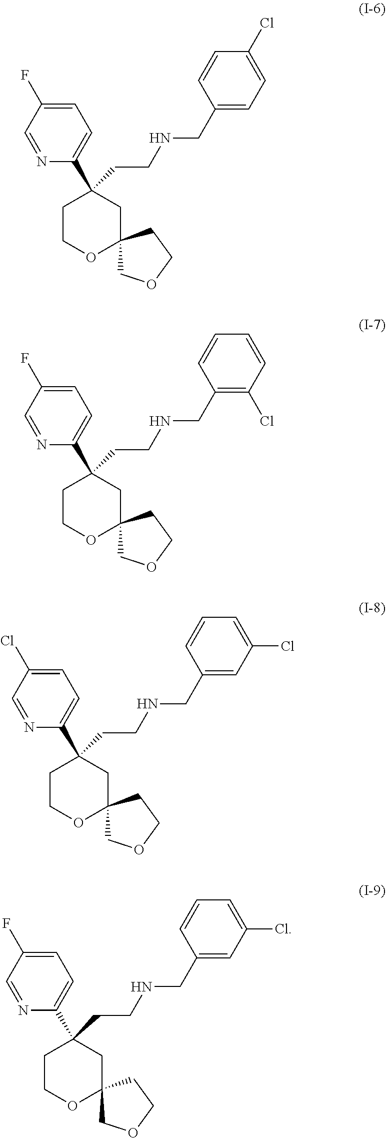 [SMILES: Fc1ccc([C@]2(CCNCc3ccc(Cl)cc3)CCO[C@]3(CCOC3)C2)nc1 ; Fc1ccc([C@]2(CCNCc3ccccc3Cl)CCO[C@]3(CCOC3)C2)nc1 ; Clc1ccc([C@]2(CCNCc3cccc(Cl)c3)CCO[C@]3(CCOC3)C2)nc1 ; Fc1ccc([C@@]2(CCNCc3cccc(Cl)c3)CCO[C@@]3(CCOC3)C2)nc1]

In some embodiments, compound (a) is selected from a stereoisomer or mixture of stereoisomers.

In some embodiments, compound (a) is selected from:

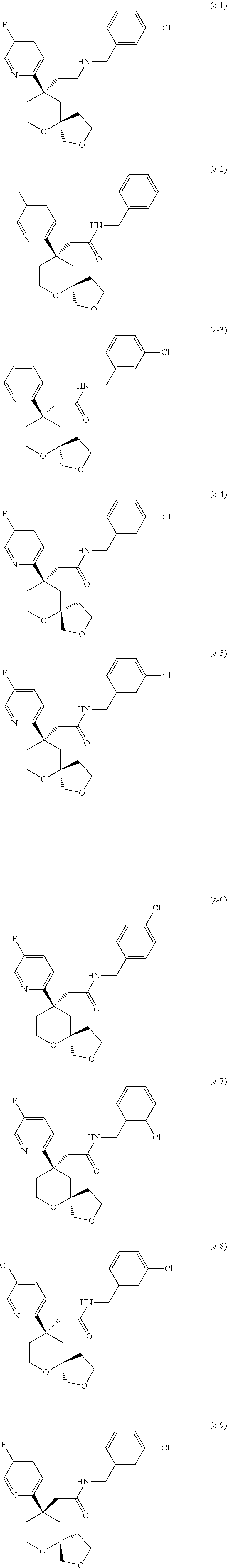

In some embodiments, said compound (I) is compound (I-1) and compound (a) is compound (a-1); the preparation method of compound (I-1) or its salt comprises:

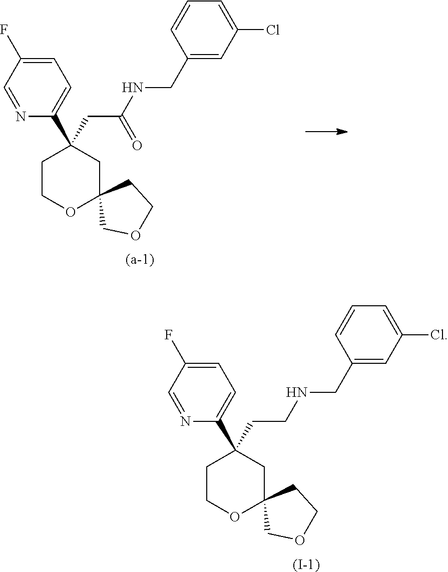

In some embodiments, in any of the above-mentioned preparation methods of compound (I), NaBH₄—ZnCl₂-triethylamine-protic solvent was used as the reducing system; the ZnCl₂ was preferably anhydrous ZnCl₂.

In some embodiments, in any of the above-mentioned preparation methods of compound (I), the protic solvent of the reducing system is selected from methanol, ethanol, isopropanol, n-butanol, formic acid or acetic acid, preferably methanol.

In some embodiments, in any of the above-mentioned preparation methods of compound (I), the molar ratio of sodium borohydride (NaBH₄) to compound (a) is 4˜6:1, preferably 4:1; the molar ratio of zinc chloride (ZnCl₂) to compound (a) is 2˜3:1, preferably 2:1; the molar ratio of triethylamine to compound (a) is 0.5˜2:1 or 0.5˜1.5:1, preferably 1˜2:1, more preferably 1˜1.5:1, such as 1.4:1 or 1:1; the molar ratio of protic solvent to compound (a) is 1˜4.5:1, or 1˜3:1, preferably 3˜4.5:1, 2:1, 3:1, or 4:1.

In some embodiments, in any of the above-mentioned preparation methods of compound (I), the reaction solvent is one or more of ether and aromatic solvents; the ether solvents are selected from ether, methyl tert butyl ether, benzyl ether, tetrahydrofuran, 2-methyltetrahydrofuran, 1,4-dioxane, etc.; the aromatic solvents are selected from toluene, xylene, etc. The reaction solvent is preferably a mixture of tetrahydrofuran and toluene.

In some embodiments, in any of the above-mentioned preparation methods of compound (I), the reaction temperature is 75˜90° C. or 75˜85° C., preferably 80˜90° C. or 78˜82° C., and the reaction time is 2˜15 h, preferably 3˜9 h, 5˜9 h or 7˜9 h.

In some embodiments, any of the above-mentioned preparation methods of compound (I) includes the following steps: dissolving sodium borohydride in an ether solvent, cooling, adding anhydrous zinc chloride, and reacting for a certain time; successively adding compound (a), aromatic hydrocarbon solvent, and triethylamine, adding protic solvent dropwise, performing the reduction reaction, quenching, evaporating the organic solvent under reduced pressure, adjusting the aqueous phase to pH=12˜14, extracting the aqueous phase, successively washing the organic phase with aqueous ammonium chloride solution and aqueous sodium chloride solution, and concentrating under reduced pressure to dryness to obtain the crude compound (I), which is directly used in the next step. The ether solvent is preferably tetrahydrofuran; the aromatic hydrocarbon solvent is preferably toluene; the alcohol reagent is preferably methanol; the extraction solvent is preferably one or more of a halogenated hydrocarbon solvent, aliphatic ester solvent, aromatic hydrocarbon solvent and/or ether solvent, and preferably a halogenated hydrocarbon solvent or aliphatic ester solvent.

In one aspect of the present invention, any of the above-mentioned preparation methods of compound (I) or its salt, the preparation method of compound (a) or a stereoisomer or mixture of stereoisomers comprises:

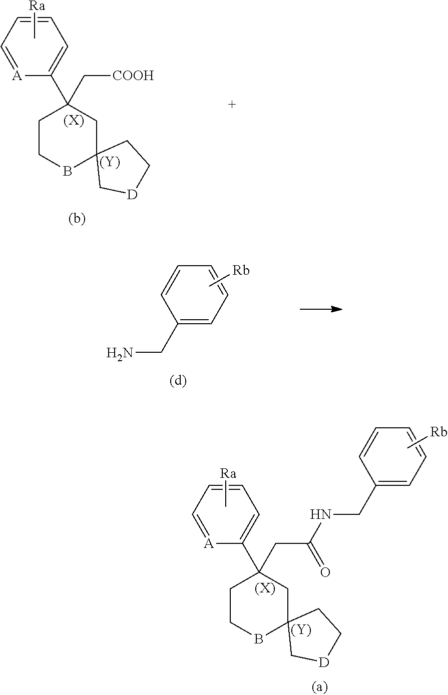

wherein, A, B, D, R^{a}, R^{b}, (X) and (Y) are defined as in compound (I); compound (d) can also be selected in the form of its salt, such as the hydrochloride salt of compound (d).

In some embodiments, compound (a) is compound (a-1), compound (b) is compound (b-A-1), and compound d is compound (d-1), wherein the preparation method of said compound (a-1) comprises:

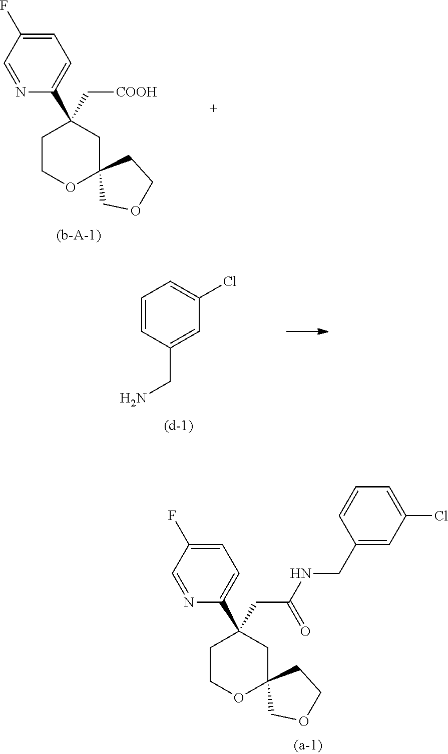 [SMILES: O=C(O)C[C@@]1(c2ccc(F)cn2)CCO[C@]2(CCOC2)C1 ; NCc1cccc(Cl)c1 ; O=C(C[C@@]1(c2ccc(F)cn2)CCO[C@]2(CCOC2)C1)NCc1cccc(Cl)c1]

wherein the compound (d-1) can be selected in the form of its salt, such as the hydrochloride salt of compound (d-1).

In some embodiments, in any of the above-mentioned preparation methods of compound (a) from compound (b), the condensing agent is selected from 1-ethyl-(3-dimethylaminopropyl) carbodiimide hydrochloride (EDCI), 2-(7-azabenzotriazole)-N,N,N′,N′-tetramethylurea hexafluorophosphate (HATU) and N,N′-carbonyldiimidazole (CDI), etc., preferably CDI; the reaction solvent is one or more of acrylic solvent, ether solvent, sulfone solvent and/or sulfoxide solvent, preferably ether solvent; the reaction temperature is 0˜50° C., preferably 20˜30° C.

In some embodiments, any of the above-mentioned preparation methods of compound (a) from compound (b) includes the following steps: dissolving compound (b) and the condensing agent in a solvent, reacting for a certain time, adding compound (d) or its salt, conducting a condensation reaction, completing the reaction, extracting and filtering, concentrating the filtrate under reduced pressure to dryness, dissolving with benign solvent, successively washing with dilute acid and aqueous sodium chloride solution, concentrating under reduced pressure to dryness, beating with poor solvent, extracting and drying to obtain compound (a). The solvent is preferably one or more of an acrylic solvent, an ether solvent, a sulfone solvent and a sulfoxide solvent; the good solvent is one or more of an aliphatic ester solvent and/or a halogenated hydrocarbon solvent; and the poor solvent is one or more of an alkane solvent, an aromatic hydrocarbon solvent and/or an ether solvent.

In some embodiments, in any of the above-mentioned preparations of compound (a) from compound (b), stereoisomers of compound (a) are prepared from compound (b-X) (a stereoisomer of compound (b)), wherein a method for preparing compound (b-X) comprises:

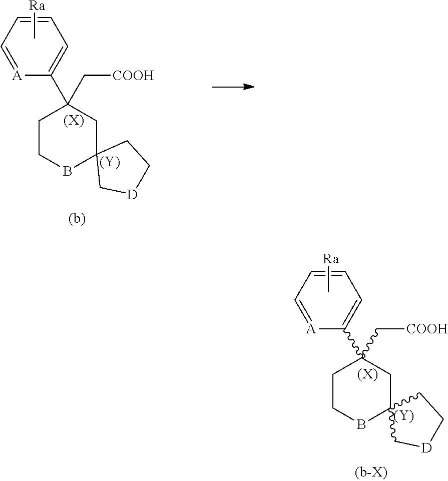

wherein, A, B, D and Ra are defined as in compound (I), preferably A is N, B and D are all O; compound (b) is a racemic compound or enantiomerically enriched form with different (X) and (Y) configurations.

In some embodiments, in any of the above-mentioned preparations of compound (b-X) from compound (b), the compound (b) is enantiomerically enriched with different (X) and (Y) configurations. Preferably, compound (b) comprises compound (b-A) with configuration (5S, 9R) and compound (b-B) with configuration (5R, 9S), and the combined weight content of compound (b-A) or compound (b-B) in compound (b) is about 95% or more, preferably more than about 98%.

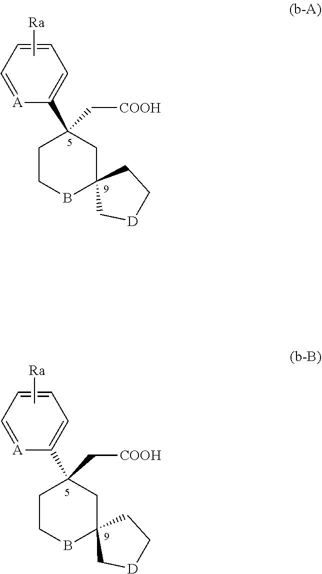

The term “enantiomerically enriched” in the present invention means that the content of said enantiomers is 60% or more, 70% or more, 80% or more, 90% or more, 95% or more, 96% or more, 97% or more, 98% or more, or 99% or more.

In some embodiments, in any of the above-mentioned preparations of compound (b-X) from compound (b), compound (b) is compound (b-1), compound (b-X) is compound (b-A-1), and the method for preparing compound (b-X) comprises:

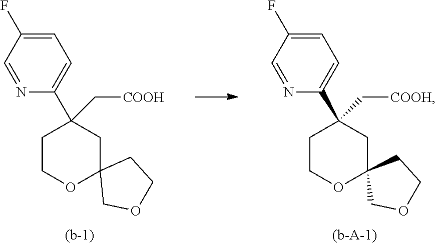 [SMILES: O=C(O)CC1(c2ccc(F)cn2)CCOC2(CCOC2)C1 ; O=C(O)C[C@@]1(c2ccc(F)cn2)CCO[C@]2(CCOC2)C1]

compound (b-1) is a racemic compound or a mixture of enriched compounds (b-A-1) and compounds (b-B-1).

In some embodiments, in any of the above-mentioned preparations of compound (b-X) from compound (b), the chemical resolution reagent is selected from cinchonidine or L-phenylethylamine, and the solvent is selected from methanol-water solvent mixture, N, N-dimethylformamide (DMF)-water solvent mixture, or N-methylpyrrolidone (NMP)-water solvent mixture.

In some embodiments, in any of the above-mentioned preparations of compound (b-X) from compound (b), the reaction temperature is 40 to 70° C. and preferably 55 to 65° C.

In some embodiments, any of the above-mentioned preparations of compound (b-X) from compound (b) includes the following steps: dissolving cinchonidine or levophenylethylamine in solvent, dissolving compound b in solvent, adding compound b solution dropwise to cinchonidine or levophenylethylamine solution, and performing an acid-base resolution reaction to obtain cinchonidine or levophenylethylamine salt of compound b-X, then the cinchonidine or levophenylethylamine salt of compound b-X is dissociated by sodium hydroxide or potassium hydroxide, the solid solution is separated, the filtrate is adjusted to pH=3˜4, the aqueous phase is extracted, concentrated to dryness under reduced pressure, aliphatic or halohydrocarbon solvents are added, crystals are stirred, alkane or aromatic hydrocarbon solvents or ether solvents are added, crystals are stirred continuously, and then filtered to obtain the compound shown in compound b-X. The solvent is selected from one or more of the methanol-water solvent mixture, DMF-water solvent mixture or NMP-water solvent mixture; the extraction solvent is selected from one or more of a halogenated hydrocarbon solvent, aliphatic ester solvent, aromatic hydrocarbon solvent and/or ether solvent, wherein a halogenated hydrocarbon solvent and/or aliphatic ester solvent are preferred.

In one aspect of the present invention, a preparation method of compound (b), a stereoisomer, or a mixture of its stereoisomers is provided, comprising:

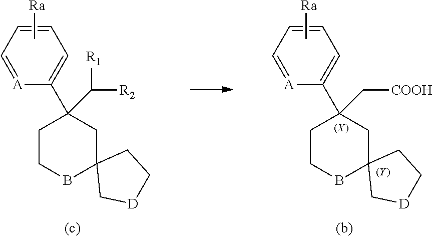

Wherein, R₁ is selected from a cyanyl, amide, ester, carboxyl, or acyl halide group;
   R₂ is selected from a hydrogen, cyanyl, amide, ester, carboxyl, or acyl halide group;
   A is selected from C or N; B and D are independently selected from C, N, or O;
   Ra is selected from hydrogen, fluorine, chlorine, bromine, or iodine atoms; and the carbon atoms with (X) and (Y) are chiral carbon atoms.

In some embodiments, in the above-mentioned preparation method of compound (b) from compound (c), A in compound (b) and compound (c) is N; and both B and D are O.

In some embodiments, in any of the above-mentioned preparation methods of compound (b) from compound (c), A in compound (b) and compound (c) is N; both B and D are O; and Ra is F.

In some embodiments, in any of the above-mentioned preparation methods of compound (b) from compound (c), compound (b) is a racemic compound or enantiomerically enriched form with different (X) and (Y) configurations.

In some embodiments, in any of the above-mentioned preparations of compound (b) from compound (c), compound (b) is enantiomerically enriched with different (X) and (Y) configurations. Preferably, compound (b) comprises compound (b-A) with configuration (5S, 9R) and compound (b-B) with configuration (5R, 9S), and the combined weight content of compound (b-A) and compound (b-B) in compound (b) is about 95% or more, preferably more than about 98%.

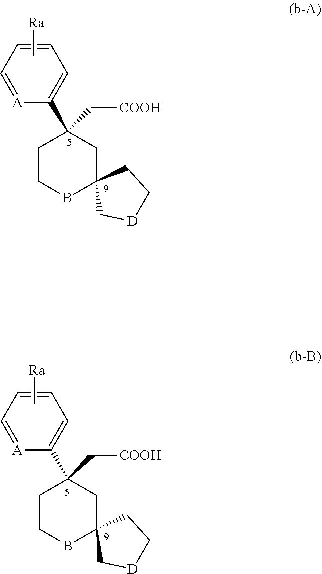

In some embodiments, in any of the above-mentioned preparations of compound (b) from compound (c), the weight ratio of compound (b-A) to compound (b-B) in compound (b) is about 1:1.

In some embodiments, in any of the above-mentioned preparations of compound (b) from compound (c), compound (b) is compound (b-1), compound (c) is compound (c-1), and the preparation method of compound (b-1) comprises:

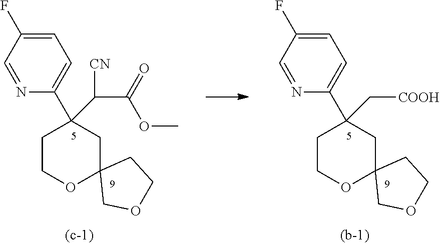 [SMILES: COC(=O)C(C#N)C1(c2ccc(F)cn2)CCOC2(CCOC2)C1 ; O=C(O)CC1(c2ccc(F)cn2)CCOC2(CCOC2)C1]

In some embodiments, in any of the above-mentioned preparations of compound (b-1) from compound (c-1), compound (b-1) is a mixture of enriched compound (b-A-1) with configuration (5S, 9R) and compound (b-B-1) with configuration (5R, 9S), and the combined weight content of compound (b-A-1) and compound (b-B-1) in compound (b) is about 95% or more, preferably more than about 98%.

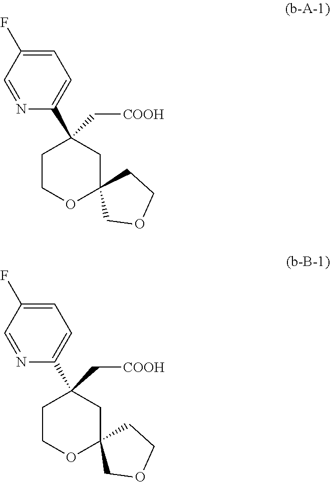 [SMILES: O=C(O)C[C@@]1(c2ccc(F)cn2)CCO[C@]2(CCOC2)C1 ; O=C(O)C[C@]1(c2ccc(F)cn2)CCO[C@@]2(CCOC2)C1]

In some embodiments, in any of the above-mentioned preparations of compound (b) from compound (c), the weight ratio of compound (b-A-1) to compound (b-B-1) in compound (b) is about 1:1.

In some embodiments, in any of the above-mentioned preparations of compound (b) from compound (c), R₁ in compound (c) is selected from cyanyl or —COOC₁₋₄ alkyl, preferably —COOCH₃; and R₂ is selected from hydrogen or cyanyl.

In some embodiments, in any of the above-mentioned preparations of compound (b) from compound (c), R₁ in compound (c) is selected from compound (c-1) or compound (c-2):

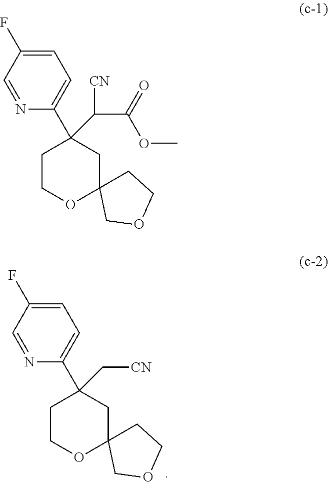 [SMILES: COC(=O)C(C#N)C1(c2ccc(F)cn2)CCOC2(CCOC2)C1 ; N#CCC1(c2ccc(F)cn2)CCOC2(CCOC2)C1]

In some embodiments, in any of the above-mentioned preparations of compound (b) from compound (c), an acid or base is used as a hydrolyzing agent. The acid is selected from sulfuric acid, methanesulfonic acid, p-toluenesulfonic acid, camphor sulfonic acid, hydrochloric acid, oxalic acid and trifluoroacetic acid, preferably sulfuric acid or methanesulfonic acid; the base is selected from sodium hydroxide or potassium hydroxide. An enantiomeric enrichment with different (X) and (Y) configurations can be obtained by acid hydrolysis as described, greatly reducing the content of compounds with the same (X) and (Y) configurations; while sodium hydroxide or potassium hydroxide is used for hydrolysis; the resulting product is a racemic compound with four stereoisomers.

In some embodiments, in any of the above-mentioned preparations of compound (b) from compound (c), the molar ratio of methanesulfonic acid to compound (c) is 2˜6:1, and the molar ratio of sulfuric acid to compound (c) is 1˜6:1.

In some embodiments, in the process of preparing compound (b) from compound (c), the reaction solvent is one or more of water or benzene solvents, preferably a mixture of toluene and water; the reaction temperature is 50 to 100° C., preferably 80 to 90° C.

In some embodiments, the process of preparing compound (b) from compound (c) includes the following steps: dissolving the compound (c) and the acid or base used as a hydrolyzing agent in solvent, carrying out hydrolysis reaction, completing the reaction, separating the liquid, adjusting the aqueous phase to pH=3˜4, extracting the aqueous phase, concentrating under reduced pressure to dryness, adding aliphatic ester solvent or halohydrocarbon solvent, stirring to separate solid, then adding alkane solvent or aromatic hydrocarbon solvent or ether solvent, continuing stirring to separate crystals, and extracting and drying to obtain compound as shown in formula (b); wherein the solvent is selected from one or more of water or aromatic hydrocarbon solvent; the extraction solvent is selected from one or more of halohydrocarbon solvent, aliphatic ester solvent, aromatic hydrocarbon solvent and/or ether solvent, wherein halohydrocarbon solvent and/or aliphatic ester solvent are preferred.

In one aspect of the present invention, any of the preparation methods of compound (I) or its stereoisomer, mixture of stereoisomers, or its salt further comprises any of the above-mentioned preparation procedures of compound (b) or a stereoisomer or mixture of stereoisomers.

In one aspect of the present invention, a method for preparing compound (I) or a stereoisomer, a mixture of stereoisomers, or a salt thereof is provided, wherein the method

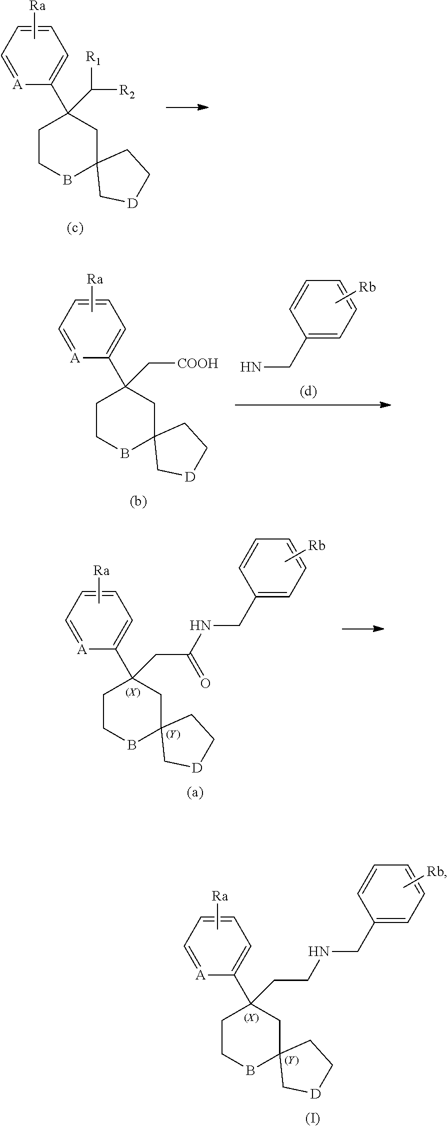

wherein, R₁ is selected from a cyanyl, amide, ester, carboxyl, or acyl halide group;
R₂ is selected from a hydrogen, cyanyl, amide, ester, carboxyl, or acyl halide group;
A is selected from C or N; B and D are independently selected from C, N, or O;
Ra and Rb are independently selected from hydrogen, fluorine, chlorine, bromine, or iodine atoms; and
the carbon atoms with (X) and (Y) are chiral carbon atoms.

In some embodiments, to obtain stereoisomers of compound (I), compound (b) is prepared into its stereoisomer (b-X), followed by further preparation of stereoisomers of compound (a) and compound (I).

In some embodiments, in any of the above-mentioned preparation methods of compound (I) from compound (a), NaBH₄—ZnCl₂-triethylamine-protic solvent was used as the reducing system; the ZnCl₂ was preferably anhydrous ZnCl₂; the protic solvent of the reducing system is selected from methanol, ethanol, isopropanol, n-butanol, formic acid or acetic acid, preferably methanol.

In some embodiments, in any of the above-mentioned preparations of compound (b) from compound (c), an acid or base is used as a hydrolyzing agent. The acid is selected from sulfuric acid, methanesulfonic acid, p-toluenesulfonic acid, camphor sulfonic acid, hydrochloric acid, oxalic acid and trifluoroacetic acid, preferably sulfuric acid or methanesulfonic acid; the base is selected from sodium hydroxide or potassium hydroxide.

In some embodiments, a method for preparing compound (I-1) or its salt is provided, wherein the method comprises:

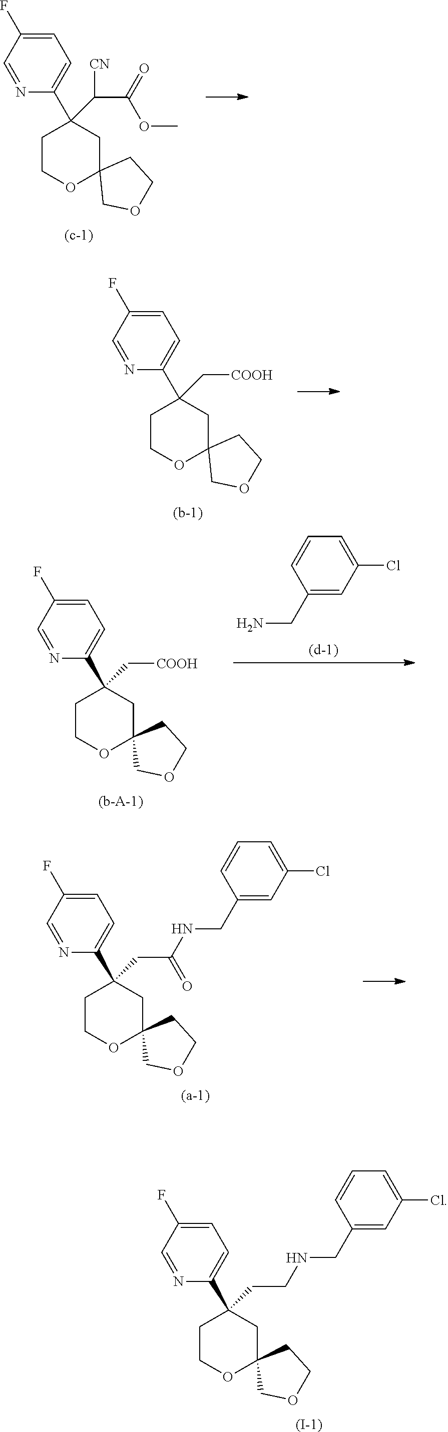

In one aspect of the present invention, a mixture comprising compound (I-1) or a salt thereof has impurity B (i.e., compound (I-3)) in a weight amount of less than about 0.5%, preferably less than about 0.3%, more preferably less than 0.15%.

In one aspect of the present invention, a mixture comprising compound (I-1) or a salt thereof has impurity F in a weight amount of less than about 0.5%, preferably less than about 0.4%, more preferably less than 0.3%; wherein the structural formula of impurity F is

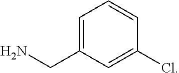

In some embodiments, the salts of compound (I-1) are selected from malate, maleate, fumarate, hydrochloride, tartrate, phosphate or citrate, etc.

In some embodiments, the present invention further provides a mixture comprising maleic acid salt of compound (I-1), wherein impurity B is present with a weight content of less than about 0.5%, preferably less than about 0.3%, more preferably less than 0.15%.

In some embodiments, the present invention further provides a mixture comprising a maleic acid salt of compound (I-1), wherein impurity F is present with a weight content of less than 0.5%, preferably less than about 0.4%, more preferably less than 0.3%.

In one aspect of the present invention, a preparation method of compound (I) salt is provided, comprising dissolving compound (I) in an organic solvent, ethyl acetate, adding an acid under certain conditions, and forming a salt of compound (I) with an acid.

In one aspect of the present invention, compound (a) or a stereoisomer or a mixture of stereoisomers is provided:

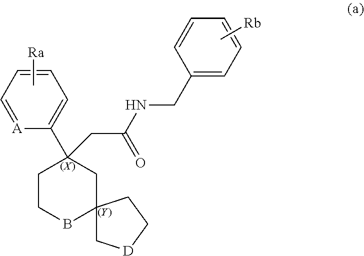

wherein,
A is selected from C or N;
B or D independently selected from C, N, or O;
Ra or Rb are independently selected from hydrogen, fluorine, chlorine, bromine, or iodine atoms; and the carbon atoms with (X) and (Y) are chiral carbon atoms.

In some embodiments, A is N; B and D are both O.

In some embodiments, A is N; B and D are both O, Ra is fluorine atom; and Rb is chlorine atom.

In some embodiments, the configurations of (X) and (Y) in compound (a) are the same or different. Preferably, the configuration of (X) in compound (a) is S, and the configuration of (Y) is R; or the configuration of (X) is R, and the configuration of (Y) is S.

In some embodiments, compound (a) is selected from:

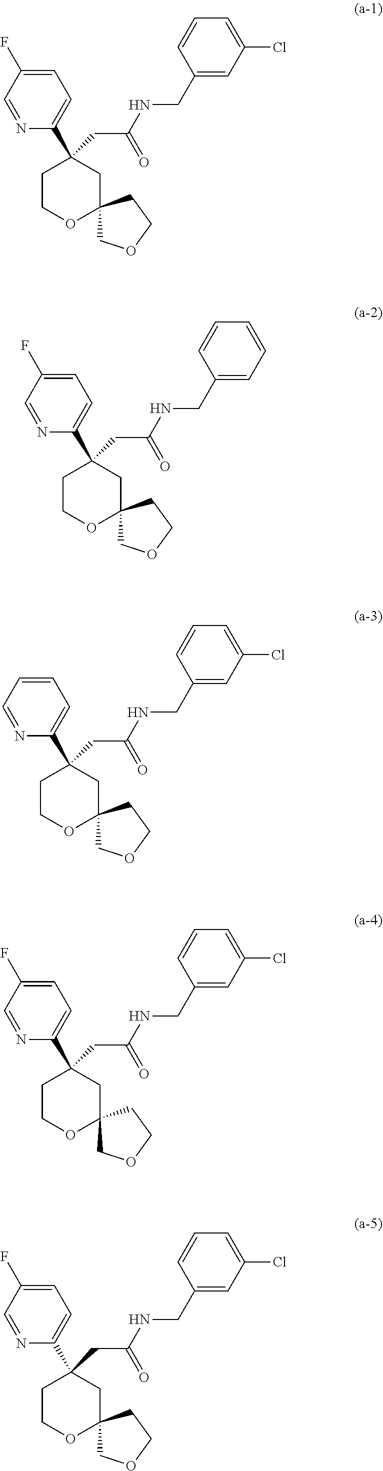 [SMILES: O=C(C[C@@]1(c2ccc(F)cn2)CCO[C@]2(CCOC2)C1)NCc1cccc(Cl)c1 ; O=C(C[C@@]1(c2ccc(F)cn2)CCO[C@]2(CCOC2)C1)NCc1ccccc1 ; O=C(C[C@@]1(c2ccccn2)CCO[C@]2(CCOC2)C1)NCc1cccc(Cl)c1 ; O=C(C[C@@]1(c2ccc(F)cn2)CCO[C@@]2(CCOC2)C1)NCc1cccc(Cl)c1 ; O=C(C[C@]1(c2ccc(F)cn2)CCO[C@]2(CCOC2)C1)NCc1cccc(Cl)c1]

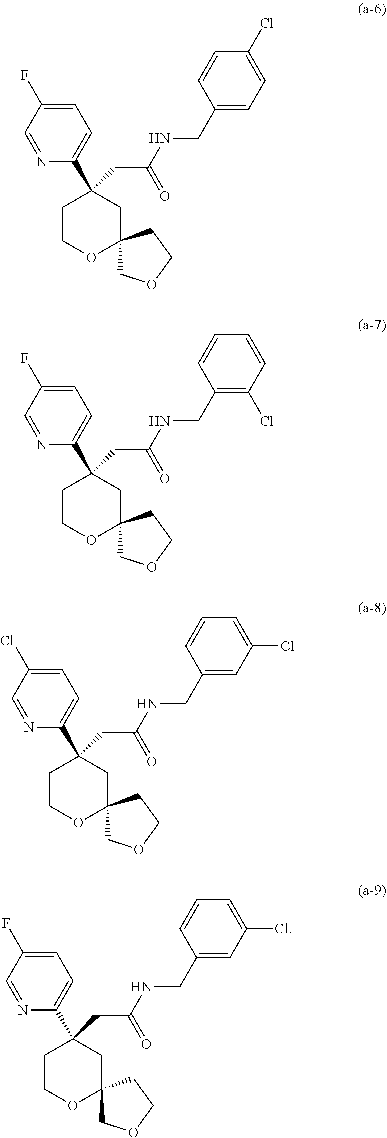 [SMILES: O=C(C[C@@]1(c2ccc(F)cn2)CCO[C@]2(CCOC2)C1)NCc1ccc(Cl)cc1 ; O=C(C[C@@]1(c2ccc(F)cn2)CCO[C@]2(CCOC2)C1)NCc1ccccc1Cl ; O=C(C[C@@]1(c2ccc(Cl)cn2)CCO[C@]2(CCOC2)C1)NCc1cccc(Cl)c1 ; O=C(C[C@]1(c2ccc(F)cn2)CCO[C@@]2(CCOC2)C1)NCc1cccc(Cl)c1]

In one aspect of the present invention, compound (b) or a stereoisomer or a mixture of stereoisomers is provided:

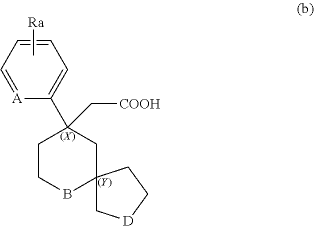

wherein,
A is selected from C or N, and B or D is independently selected from C, N, or O; preferably A is N; and B and D are O;
Ra is selected from hydrogen, fluorine, chlorine, bromine, or iodine atoms;
and the carbon atoms with (X) and (Y) are chiral carbon atoms.

In some embodiments, compound (b) is enantiomerically enriched with different (X) and (Y) configurations. Preferably, compound (b) comprises compound (b-A) with configuration (5S, 9R) and compound (b-B) with configuration (5R, 9S), and the combined weight content of compound (b-A) and compound (b-B) in compound (b) is about 95% or more, preferably more than about 98%. More preferably, the weight ratio of compound (b-A) to compound (b-B) in compound (b) is about 1:1.

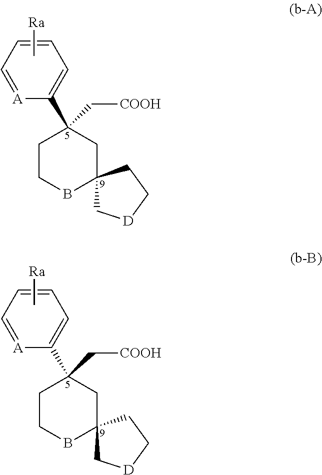

In some embodiments, compound (b) is preferably compound (b-1) or a stereoisomer, preferably compound (b-A-1) or compound (b-B-1):

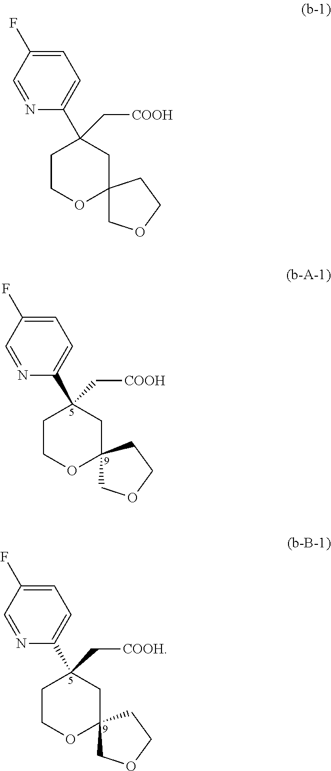

In some embodiments, compound (b-1) is enantiomerically enriched with different (X) and (Y) configurations. Preferably, compound (b-1) comprises compound (b-A-1) and compound (b-B-1), and the combined weight content of compound (b-A-1) and compound (b-B-1) in compound (b) is about 95% or more, preferably more than about 98%. More preferably, the weight ratio of compound (b-A-1) to compound (b-B-1) in compound (b) is about 1:1.

The present invention has one or more advantages and beneficial effects as follows:
1. No need for SFC separation, no need for hazardous catalytic hydrogenation reduction and cumbersome column chromatography purification;
2. The reaction conditions are mild, the purification method is simple, feasible, and environmentally friendly, suitable for industrial large-scale production;
3. The prepared compound (I) only contains a small amount of structurally similar impurities, which is convenient for further processing;
4. The conversion rate of the target compound is high, and parallel and batch scaling experiments show that the catalytic system has good process stability and controllability;
5. The intermediates are all in a solid state, which facilitates quality control of intermediates and finished products in industrial production.
6. It can significantly reduce production costs, production cycle time, waste generation, and energy consumption.
7. It can effectively reduce impurity content and improve product quality.

### DETAILED DESCRIPTION OF EMBODIMENTS

The present invention is described in detail below by way of examples. However, this is by no means disadvantageously limiting the scope of the present invention. Experimental methods that do not specify specific conditions in the implementation examples are usually carried out under conventional conditions or conditions recommended by the raw material or product manufacturer. Reagents without a specific source specified are conventional reagents purchased from the market.

The impurities in compound (I-1) and its salts were detected by high-performance liquid chromatography under the following chromatographic conditions:
Column: Waters Symmetry Shield RP18 (150*4.6 mm, 3.5 um) or equivalent column
Flow rate: 1 mL/min
Column temperature: 35° C.
Injection volume: 10 μL
Detection wavelength: 210 nm
Running time: 70 minutes

Mobile phase A: 2.5 mmol/L sodium octanesulfonate solution: weigh approximately 0.54 g of sodium octanesulfonate and 1.36 g of potassium dihydrogen phosphate in 1000 mL of purified water, add 1 mL of triethylamine, adjust the pH to 2.5±0.05 with phosphoric acid, mix well, and degas through ultrasonic filtration to obtain the desired solution
Mobile phase B: acetonitrile
Mobile phase gradient:

| Time(min) | Phase A % | Phase B % |
|---|---|---|
| 0 | 76 | 24 |
| 30 | 76 | 24 |
| 56 | 50 | 50 |
| 57 | 76 | 24 |
| 66 | 76 | 24 |

### Example 1

### Synthesis of Compound (B-1)

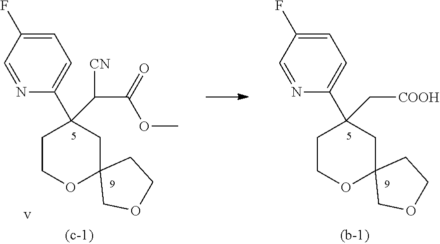 [SMILES: COC(=O)C(C#N)C1(c2ccc(F)cn2)CCOC2(CCOC2)C1 ; O=C(O)CC1(c2ccc(F)cn2)CCOC2(CCOC2)C1]

### 1. Sulfuric Acid Hydrolysis Method

At room temperature, 300 g of compound (c-1) (in which the ratio of four isomers of (5R, 9S), (5R, 9R), (5S, 9R) and (5S, 9S) was about 1:1:1:1) was measured out, toluene (1200 ml) and water (1500 ml) were added, stirred and mixed well, concentrated sulfuric acid (264 g, 2.5 eq) was slowly added, the reaction mixture was heated up to 85° C. for 4 h, then the temperature was reduced to below 30° C., separated, the aqueous phase pH was adjusted with concentrated hydrochloric acid to 3˜4, extracted with dichloromethane (3000 ml, 1500 ml×2), the organic phases were combined and concentrated under reduced pressure to dryness, ethyl acetate (300 ml) was added, the temperature was controlled at 10° C., the precipitated solid was stirred, n-heptane (1200 mL) was added, the temperature was controlled at −5° C., the crystal was stirred for 2 h then filtered, the filter cake was washed with n-heptane (300 ml) then dried to obtain white solid, which was compound (b-1) 119.2 g, yield 45.0%, purity 97.7%. The contents of (5R, 9S), (5R, 9R), (5S, 9R), and (5S, 9S) isomers in compound (b-1) were 48.91%, 0.11%, 48.86%, and 0.06%, respectively, and the compound (b-A-1) (5S, 9R) and compound (b-B-1) (5R, 9S) configuration ratios were 1:1. HRMS m/z: 296.1294 [M+1]+1. H NMR (600 MHz, CDCl₃) δ 9.11˜12.45 (br, 1H), 8.41˜8.43 (m, 1H), 7.35˜7.40 (m, 2H), 3.71˜3.89 (m, 4H), 3.52˜3.55 (d, J=9.6 Hz, 2H), 2.77˜2.80 (d, J=15 Hz, 1H), 2.52˜2.55 (d, J=15 Hz, 1H), 2.44˜2.47 (d, J=13.2 Hz, 2H), 2.00˜1.93 (m, 2H), 1.46-1.52 (m, 1H), 1.17-1.20 (m, 1H).

### 2. Methanesulfonic Acid Hydrolysis Method

At room temperature, 300 g of compound (c-1) (in which the ratio of four isomers of (5R, 9S), (5R, 9R), (5S, 9R) and (5S, 9S) was about 1:1:1:1) was measured out, toluene (11.5 kg) and water (13 kg) were added, methanesulfonic acid (5 kg, 5 eq) was slowly added, after completing the addition, the reaction mixture was heated up to 85° C. for 6 h, then reduced to below 30° C., separated, the aqueous phase was adjusted to pH=3˜4 with concentrated hydrochloric acid, then extracted with dichloromethane (22 kg4), the organic phases were combined and then concentrated under reduced pressure to dryness, ethyl acetate (3 kg) was added, the temperature was controlled at 20° C., the reaction mixture was stirred to precipitate solid, n-heptane (9 kg) was added, the reaction mixture was stirred for 3 h at controlled temperature −5° C. to crystallize, filtered, the filter cake was washed with n-heptane (2 kg), then dried to obtain a white solid of 1.26 kg, which was compound (b-1), purity of 98.8%. The (5R, 9S), (5R, 9R), (5S, 9R), and (5S, 9S) isomers in compound (b-1) were 49.21%, 0.00%, 49.36%, and 0.00%, respectively, and the compound (b-A-1) (5S, 9R) and compound (b-B-1) (5R, 9S) configuration ratios were approximately 1:1. HRMS m/z: 296.1290 [M+1]+1. H NMR (600 MHz, CDCl₃) δ 9.11˜12.45 (br, 1H), 8.41˜8.43 (m, 1H), 7.35˜7.40 (m, 2H), 3.71˜3.89 (m, 4H), 3.52˜3.55 (d, J=9.6 Hz, 2H), 2.77˜2.80 (d, J=15 Hz, 1H), 2.52˜2.55 (d, J=15 Hz, 1H), 2.44˜2.47 (d, J=13.2 Hz, 2H), 2.00˜1.93 (m, 2H), 1.46-1.52 (m, 1H), 1.17-1.20 (m, 1H).

### 3. Hydrochloric Acid Hydrolysis Method

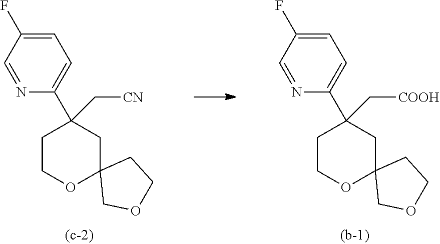 [SMILES: N#CCC1(c2ccc(F)cn2)CCOC2(CCOC2)C1 ; O=C(O)CC1(c2ccc(F)cn2)CCOC2(CCOC2)C1]

At room temperature, 780 g compound (c-2) (in which the ratio of four isomers of (5R, 9S), (5R, 9R), (5S, 9R) and (5S, 9S) was about 1:1:1:1) and water (3120 ml) were added into the reactor, concentrated hydrochloric acid (1386.1 g, 4.0 eq) was added under stirring, heated up to 85˜95° C. for reaction for 2 h, then reduced to below 35° C., washed with methyl tert-butyl ether (1560 ml), the liquid was separated, the pH was adjusted to pH=3˜4 using 30% sodium hydroxide aqueous solution, extracted with dichloromethane (3120 mL) 3 times, the organic phases were combined, washed with water (1560 ml), concentrated under reduced pressure to dryness, ethyl acetate (1170 ml) was added, temperature was controlled at 10° C., stirred to precipitate solid, n-heptane (3510 mL) was added, temperature was controlled at −5° C. while stirring for 2 h to crystallize, filtered, and the filter cake was washed with n-heptane (780 ml), dried to white solid, i.e. compound (b-1) 340.3 g, yield 40.8%, purity 98.86%. The content of (5R, 9S), (5R, 9R), (5S, 9R) and (5S, 9S) isomers in compound (b-1) was 49.68%, 0.0%, 49.70% and 0.0%, respectively, and the ratio of compound (b-A-1) (5S, 9R) and compound (b-B-1) (5R, 9S) configuration was 1:1. HRMS m/z: 296.1294 [M+1]+iₒ HNMR (600 MHz, CDCl3) δ 9.11˜12.45 (br, 1H), 8.41˜8.43 (m, 1H), 7.35˜7.40 (m, 2H), 3.71˜3.89 (m, 4H), 3.52˜3.55 (d, J=9.6 Hz, 2H), 2.77˜2.80 (d, J=15 Hz, 1H), 2.52˜2.55 (d, J=15 Hz, 1H), 2.44˜2.47 (d, J=13.2 Hz, 2H), 2.00˜1.93 (m, 2H), 1.46-1.52 (m, 1H), 1.17-1.20 (m, 1H).

### 4. Potassium Hydroxide Hydrolysis Method

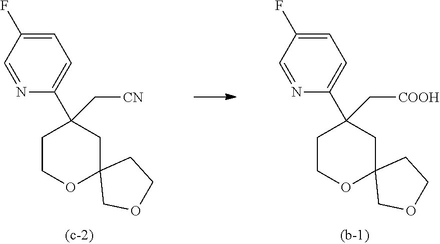 [SMILES: N#CCC1(c2ccc(F)cn2)CCOC2(CCOC2)C1 ; O=C(O)CC1(c2ccc(F)cn2)CCOC2(CCOC2)C1]

According to the above synthetic route, with reference to the methanesulfonic acid hydrolysis method, methanesulfonic acid was replaced with potassium hydroxide (KOH), and the reaction mixture was heated up to 100° C. for reaction for 30 h. Other methods were the same as the methanesulfonic acid hydrolysis method. The contents of (5R, 9S), (5R, 9R), (5S, 9R), and (5S, 9S) isomers in the obtained compound (b-1) were 20.61%, 19.20%, 20.67%, and 19.26%, respectively, and the ratio of the four stereoisomers was about 1:1:1:1.

### Example 2

### Synthesis of Compound (b-A-1)

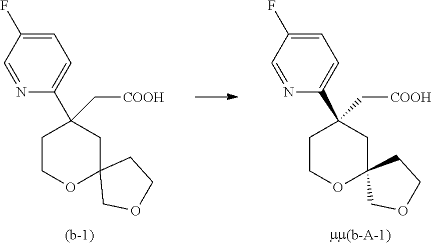 [SMILES: O=C(O)CC1(c2ccc(F)cn2)CCOC2(CCOC2)C1 ; O=C(O)C[C@@]1(c2ccc(F)cn2)CCO[C@]2(CCOC2)C1]

1. Resolution with Cinchonidine

At room temperature, cinchonidine (0.887 kg), methanol (19 kg), and water (3 kg) were added to a 50 L reaction vessel and dissolved by stirring with warming to 60° C. Compound (b-1) (0.89 kg, prepared by hydrolysis of sulfuric acid or methanesulfonic acid according to Example 1) in methanol (6.4 kg) and water (0.9 kg) was titrated and reacted at 60° C. for 2 h, then the temperature was reduced to 5° C., and the reaction mixture was allowed to precipitate for 4 h. After extraction and washing with methanol (1.4 kg), 748 g of white solid was dried. Purified water (5.4 kg) and sodium hydroxide (0.07 kg) were added into the white solid, stirred for dissolution, reacted for 1 h, extracted, washed with purified water (1.8 kg); the filtrate was combined, the pH was adjusted to 3˜4 with concentrated hydrochloric acid, the reaction mixture was stirred for 30 min, extracted with dichloromethane (5.3 kg×4); the organic phases were combined, concentrated to dryness under reduced pressure, ethyl acetate (2.9 kg) was added, a white solid was precipitated by stirring, n-heptane (5.5 kg) was added, and stirred and allowed to crystallize for 3 h; filtered under vacuum; the filter cake was washed with n-heptane (0.6 kg), and dried to 325 g of white solid compound (b-A-1). Overall yield 36.5%, chemical purity 99.93%, optical purity 99.82%. HRMS m/z: 296.1290 [m+1]+1. H NMR (600 MHz, CDCl3) δ8.40 (dd, J=0.8, 2.8 Hz, 1H), 7.31-7.39 (m, 2H), 3.66-3.87 (m, 5H), 3.51 (d, J=9.2 Hz, 1H), 2.76 (d, J=14.8 Hz, 1H), 2.51 (d, J=14.8 Hz, 1H), 2.44 (dd, J=1.2, 13.6 Hz, 1H), 2.43-2.48 (m, 1H), 1.96 (d, J=14.0 Hz, 1H), 1.88-1.96 (m, 1H), 1.43-1.49 (m, 1H), 1.12-1.25 (m, 1H).

2. Resolution with L-Phenylethylamine

At room temperature, compound (b-1) (10 g, prepared by hydrolysis of sulfuric acid or methanesulfonic acid according to Example 1) was added into acetone (100 ml), stirred to dissolve; levophenylethylamine (4 g) in acetone (50 ml) solution was added dropwise, reacted at room temperature for 1 h, filtered to obtain white solid 3.3 g; water (30 ml) was added to white solid, stirred, the pH was adjusted to 1˜2 with hydrochloric acid until the reaction solution clearly dissolves; the pH was adjusted to 3˜4 with sodium hydroxide solution, stirred to react for 30 min, extracted with dichloromethane (30 ml×4), and the organic phases were combined. Solid compound (b-A-1) 1.8 g was obtained by vacuum concentration in 18% overall yield, 98.83% chemical purity, and 94.93% optical purity. HRMS m/z: 296.1290 [m+1]+1. H NMR (600 MHz, CDCl₃) δ8.40 (dd, J=0.8, 2.8 Hz, 1H), 7.31-7.39 (m, 2H), 3.66-3.87 (m, 5H), 3.51 (d, J=9.2 Hz, 1H), 2.76 (d, J=14.8 Hz, 1H), 2.51 (d, J=14.8 Hz, 1H), 2.44 (dd, J=1.2, 13.6 Hz, 1H), 2.43-2.48 (m, 1H), 1.96 (d, J=14.0 Hz, 1H), 1.88-1.96 (m, 1H), 1.43-1.49 (m, 1H), 1.12-1.25 (m, 1H).

### Example 3

### Synthesis of Compound (a-1)

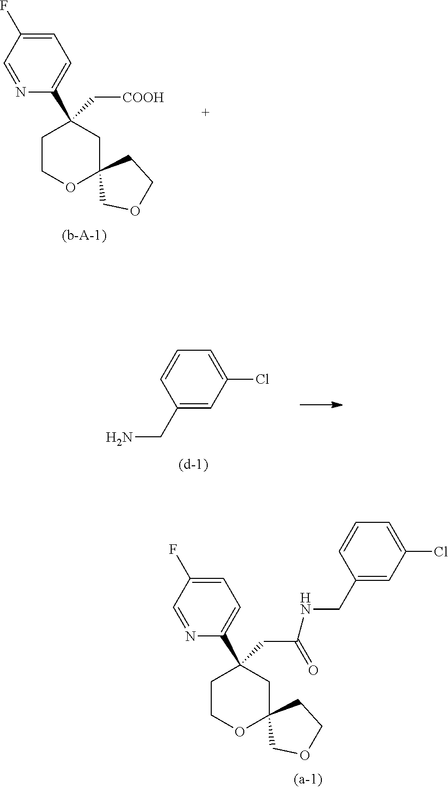 [SMILES: O=C(O)C[C@@]1(c2ccc(F)cn2)CCO[C@]2(CCOC2)C1 ; NCc1cccc(Cl)c1 ; O=C(C[C@@]1(c2ccc(F)cn2)CCO[C@]2(CCOC2)C1)NCc1cccc(Cl)c1]

Compound (b-A-1) (260 g), tetrahydrofuran (2.4 kg), and N,N′-carbonyldiimidazole (159 g) were added to a 1 L three-mouthed bottle for 1.5 h at room temperature. 3-Chlorobenzylamine (d-1) hydrochloride (191 g) was added and allowed to react for 4 hours. Filtered under vacuum, the filter cake was washed with tetrahydrofuran (0.5 kg), the filtrate was concentrated to dryness under reduced pressure, dissolved with ethyl acetate (2.3 kg), washed successively with 0.2 N aqueous hydrochloric acid solution (1 kg×3), and saturated aqueous sodium chloride solution (1 kg), concentrated to dryness under reduced pressure; n-heptane (3.3 kg) was added, and beat for 5 hours. After extraction and washing the filter cake with n-heptane (0.4 kg), 335 g of the white solid compound (a-1) was dried in 91% yield, 99.89% chemical purity, and 99.81% optical purity. HRMS m/z: 419.1532, 421.1517 [m+1]+1. H NMR (600 MHz, CDCl₃) δ8.34 (t, J=1.6 Hz, 1H), 7.27 (d, J=1.6 Hz, 1H), 7.25 (m, 1H), 7.19 (m, 1H), 7.15 (m, 1H), 6.92-6.93 (m, 1H), 6.79-6.81 (m, 1H), 5.47 (m, 3H), 4.17 (dd, J=6.8, 14.8 Hz, 1H), 3.99 (dd, J=5.6, 14.8 Hz, 1H), 3.83-3.88 (m, 1H), 3.65-3.78 (m, 1H), 3.53 (d, J=9.2 Hz, 1H), 2.62 (d, J=13.2 Hz, 1H), 2.48-2.54 (m, 1H), 2.42 (dd, J=1.6, 13.6 Hz, 1H), 2.40 (d, J=13.6 Hz, 1H), 2.00˜2.08 (m, 1H), 1.97 (d, J=13.6 Hz, 1H), 1.42-1.48 (m, 1H), 1.12-1.25 (m, 1H).

### Example 4

### Synthesis of Compounds (a-2), (a-3) and (a-8)

Compounds (a-2), (a-3) and (a-8) were prepared by referring to the methods of Example 3.

The test results are showed shown in Table 1.

TABLE 1
Compounds (a-1), (a-3) and (a-8) Characterization

| NO. | Structural Formula | ¹H NMR | MS |
|---|---|---|---|
| (a-2) | 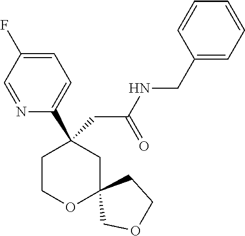 [SMILES: O=C(C[C@@]1(c2ccc(F)cn2)CCO[C@]2(CCOC2)C1)NCc1ccccc1] | DMSO-d6, δ 8.49(d, J = 4.0 Hz, 1H), 8.08(t, J = 4.0 Hz, 1H), 7.60(m, 1H), 7.49(m, 1H), 7.22(m, 3H), 6.93(d, J = 8.0 Hz, 2H), 3.97-4.15(m, 2H), 3.52- 3.73(m, 5H), 3.42(d, J = 8.0 Hz, 1H), 3.34(s, 1H), 2.50(s, 1H), 2.31-2.39(m, 2H), 2.0(d, J = 12.0 Hz, 1H), 1.86-1.91 (m, 1H), 1.30-1.33(m, 1H), 1.05-1.11 (m, 1H). | [M + H]⁺ 385.4 |
| (a-3) | 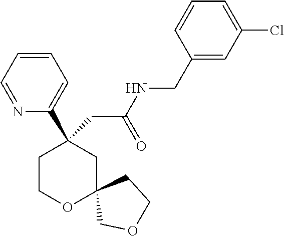 [SMILES: O=C(C[C@@]1(c2ccccn2)CCO[C@]2(CCOC2)C1)NCc1cccc(Cl)c1] | CDCl3, δ 8.52 (d, J = 4.0 Hz, 1H), 7.58- 7.60(m, 1H), 7.28-7.31 (m, 1H), 7.10- 7.19(m, 3H), 6.81-6.88(m, 2H), 4.13(dd, J = 4.0 Hz, 1H), 4.01(dd, J = 4.0 Hz, 1H), 3.87-3.98(m, 2H), 3.68-3.82(m, 3H), 3.56 (d, J = 8.0 Hz, 1H), 2.67(d, J = 16.0 Hz, 1H), 2.42-2.51(m, 3H), 2.07-2.10(m, 1H), 2.01(d, J = 12.0 Hz, 1H), 1.17-1.21(m, 1H). | [M + H]⁺ 401.4 |
| (a-8) | 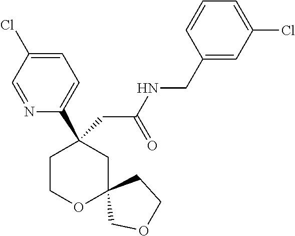 [SMILES: O=C(C[C@@]1(c2ccc(Cl)cn2)CCO[C@]2(CCOC2)C1)NCc1cccc(Cl)c1] | CDCl3, δ 8.47(s, 1H), 7.53-7.56(m, 1H), 7.17- 7.27(m, 3H), 6.99(s, 1H), 6.78-6.81(m, 1H), 5.55(s, 1H), 4.17-4.22(m, 1H), 3.99-4.04(m, 1H), 3.84-3.88(m, 2H), 3.70-3.79(m, 3H), 3.55 (d, J = 8.0 Hz, 1H), 2.66(d, J = 16.0 Hz 1H), 2.46(s, 1H), 2.40-2.43(m, 2H), 1.97-2.08(m, 2H), 1.18-1.23(m, 1H). | [M + H]⁺ 435.3 |

### Example 5

### Synthesis of Compound (I-1)

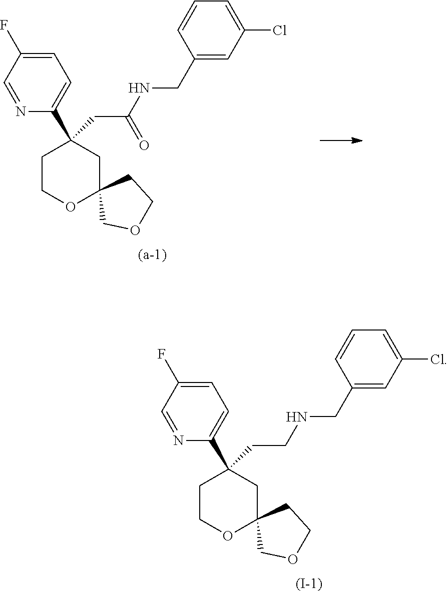

### 1. Reduction Reaction

A comparative study was conducted on the preparation methods of different reduction systems. After the reaction, the remaining amount of compound (a-1), the content of compound (I-1), impurity B, and impurity F in the reaction solution were assayed. The specific experimental conditions and results are shown in Table 2.

Tests 1 through 5 shall be prepared according to the following method: At room temperature, the reaction vessel was replaced with nitrogen gas, followed by the addition of tetrahydrofuran (25 ml), sodium borohydride (4 eq), glacial acetic acid or methanesulfonic acid, iodine or trifluoroacetic acid, or hexahydrate NiCl₂/triethylamine or LiCl/triethylamine (see Table 2). Compounds (a-1) (5 g) and toluene (25 ml) were added, and the reaction was carried out at high temperature (see Table 2 of reaction temperature/time). After the reaction, the content of each substance in the reaction solution was measured; then, the temperature was controlled below 10° C., 5 g of water was added dropwise to quench the reaction, the organic solvent was evaporated under reduced pressure, 10 g of water were added to the concentrated solution, the temperature was controlled below 10° C., the pH was adjusted to 12-14 with 30% potassium hydroxide, and the reaction mixture was extracted 3 times with ethyl acetate (2 ml×3). The organic phases were combined and concentrated under reduced pressure to obtain crude oil-like compound (I-1).

The sixth experiment was prepared using the following method: At room temperature, the reaction vessel was replaced with nitrogen, and tetrahydrofuran (1 kg) and sodium borohydride (89 g) were added. The temperature was controlled below 10° C., and anhydrous zinc chloride (156 g) was added. After adding, the temperature was raised to 25° C., and the reaction was stirred for 3 hours; Compound (a-1) (240 g), toluene (1 kg), and triethylamine (81 g) were added sequentially. After completion, the temperature was raised to 80° C., and the system began to change color. The reaction lasted for 9 hours. After the reaction, the content of each substance in the reaction solution was measured, and the temperature was controlled below 10° C. Water (240 g) was added dropwise to quench the reaction, and the organic solvent was evaporated under reduced pressure. Water (480 g) was added to the concentrated solution, and the temperature was controlled below 10° C. The pH was adjusted to 12-14 using 30% potassium hydroxide. The reaction mixture was extracted 3 times with ethyl acetate (2 kg×3). The organic phases were combined and concentrated under reduced pressure to obtain crude oil-like compound (I-1).

The seventh to ninth experiments were prepared according to the following method: At room temperature, the reaction vessel was replaced with nitrogen, and tetrahydrofuran (1 kg) and sodium borohydride (89 g) were added. The temperature was controlled below 10° C., and anhydrous zinc chloride (156 g) was added. After adding, the temperature was raised to 25° C., and the reaction was stirred for 3 hours; Compound (a-1) (240 g), toluene (1 kg), and triethylamine (81 g) were added sequentially. Methanol (37 g) was slowly added dropwise, and the temperature was raised to 80° C. The system color changed and the reaction time began to be recorded. The reaction lasted for 5˜9 hours. After the reaction, the content of each substance in the reaction solution was measured, and the temperature was controlled below 10° C. Water (240 g) was added dropwise to quench the reaction, and the organic solvent was evaporated under reduced pressure. Water (480 g) was added to the concentrated solution, and the temperature was controlled below 10° C., the pH was adjusted to 12-14 with 30% potassium hydroxide and the reaction mixture was extracted 3 times with ethyl acetate (2 kg×3). The organic phases were combined and concentrated under reduced pressure to obtain crude oil-like compound (I-1).

Impurity B is similar to compound (I-1) in structure, and there is no significant difference in its solubility in different solvents. Once impurity B is formed in the process of preparing compound (I-1), it is difficult to remove through post-processing steps; while impurity F is quite different from compound (I-1) in structure and is easy to remove. Therefore, in order to obtain a highly pure compound (I-1), the production of impurity B needs to be eliminated from the source. The experimental results showed that NaBH₄—ZnCl₂-triethylamine-methanol as a reduction system could effectively control the formation of impurity B with a fast reaction rate and high conversion rate.

TABLE 2
Experimental Methods and Results for Different Reduction Systems

| | | | Reaction | | Remaining | Compound | | | |
|---|---|---|---|---|---|---|---|---|---|
| | Reduction | | temperature/ | | amount of | (I-1) | Impurity B | Impurity F | |
| NO. | system* | Solvent | time | | compound (a-1) | Content | content | content | Conclusion |
| 1 | 4.0 eq NaBH₄ | 5.0 ml/g THF | 75° | C. | 89.96% | 0 | 0 | 3.75% | No target product |
| | 2.0 eq | 5.0 ml/g | 15 | h | | | | | formed, reaction |
| | methanesulfonic | toluene | | | | | | | failed |
| | acid | | | | | | | | |
| 2 | 4.0 eq NaBH₄ | 5.0 ml/g THF | 75° | C. | 5.17% | 72.77% | 0 | 5.59% | High conversion, |
| | 2.0 eq iodine | 5.0 ml/g | 19.5 | h | | | | | slow reaction rate |
| | | toluene | | | | | | | and long reaction |
| | | | | | | | | | time |
| 3 | 4.0 eq NaBH₄ | 10.0 ml/g THF | 65° | C. | 5.14% | 44.65% | 11.18% | 2.53% | Low conversion rate |
| | 4.0 eq TFA | | 8 | h | | | | | and high impurity |
| | | | | | | | | | B content |
| 4 | 4.0 eq NaBH₄ | 5.0 ml/g THF | 80° | C. | 95.54% | 0 | 0 | 0.30% | No target product |
| | 2.0 eq | 5.0 ml/g | 7 | h | | | | | formed, reaction |
| | hexahydrate | toluene | | | | | | | failed |
| | NiCl₂ | | | | | | | | |
| | 1.4 eq TEA | | | | | | | | |
| 5 | 4.0 eq NaBH₄ | 5.0 ml/g THF | 80° | C. | 97.43% | 0 | 0 | 0 | No target product |
| | 2.0 eq LiCl | 5.0 ml/g | 10.5 | h | | | | | formed, reaction |
| | 1.4 eq TEA | toluene | | | | | | | failed |
| 6 | 4.0 eq NaBH₄ | 5.0 ml/g THF | 80° | C. | 7.7% | 45.1% | 0.80% | 22.4% | Low conversion |
| | 2.0 eq ZnCl₂ | 5.0 ml/g | 9 | h | | | | | rate and high |
| | 1.4 eq TEA | toluene | | | | | | | impurity B |
| | | | | | | | | | content |
| 7 | 4.0 eq NaBH₄ | 5.0 ml/g THF | 80° | C. | 1.57% | 76.88% | 0.12% | 10.4% | High conversion |
| | 2.0 eq ZnCl₂ | 5.0 ml/g | 9 | h | | | | | rate and Low |
| | 1.4 eq TEA | toluene | | | | | | | impurity B |
| | 2.0 eq | | | | | | | | content |
| | methanol | | | | | | | | |
| 8 | 4.0 eq NaBH₄ | 5.0 ml/g THF | 80° | C. | 1.18% | 73.23% | 0.07% | 11.95% | High conversion |
| | 2.0 eq ZnCl₂ | 5.0 ml/g | 7 | h | | | | | rate and Low |
| | 1.4 eq TEA | toluene | | | | | | | impurity B |
| | 2.0 eq | | | | | | | | content |
| | methanol | | | | | | | | |
| 9 | 4.0 eq NaBH₄ | 5.0 ml/g THF | 80° | C. | 2.13% | 80.57% | 0 | 9.56% | High conversion |
| | 2.0 eq ZnCl₂ | 5.0 ml/g | 5 | h | | | | | rate and Low |
| | 1.4 eq TEA | toluene | | | | | | | impurity B |
| | 2.0 eq | | | | | | | | content |
| | methanol | | | | | | | | |

*Refers to the equivalents of triethylamine used relative to compound (a-1) (eq)

### 2. Removal of Impurity F

The crude compound (I-1) obtained from item 7 of Table 1 was dissolved in dichloromethane (3 kg), washed four times (2 kg*4) with 26% ammonium chloride aqueous solution, and washed one time (2 kg) with 26% sodium chloride aqueous solution. The organic layer was concentrated under reduced pressure until dry to obtain 180 g of oily compound (I-1) with a purity of 96.91%, an impurity B content of 0.12%, and an impurity F content of 0.31%.

### Example 6

### Synthesis of Compound (I-1)

Different protic solvents were selected, the amount of triethylamine was adjusted, and the amount of methanol was adjusted. The other reaction conditions were the same as the preparation method described in experiment number 9 of the reduction reaction in Example 4. After the reaction, the remaining amount of compound (a-1), compound (I-1), impurity B, and impurity F in the reaction solution were assayed. The experimental results are shown in Tables 3 to 5.

TABLE 3
Experimental results of different protic solvents

| | | Remaining | | | |
|---|---|---|---|---|---|
| Protic | Protic | amount of | Compound | Impurity | Impurity |
| solvent | solvent | compound | (I-1) | B | F |
| type | dosage* | (a-1) | Content | content | content |
| methanol | 4eq | 4.34% | 80.22% | ND | 7.96% |
| ethanol | 4eq | 13.87% | 74.63% | ND | 9.40% |
| isopropanol | 4eq | 10.56% | 73.13% | ND | 13.76% |
| acetic acid | 2eq | 23.95% | 68.45% | ND | 5.63% |

*Refers to the equivalents of protic solvent used relative to compound (a-1) (eq)

TABLE 4
Experimental results of different triethylamine amounts

| | Remaining | Compound | Impurity | Impurity |
|---|---|---|---|---|
| Dosage of | amount of | (I-1) | B | F |
| triethylamine * | compound (a-1) | Content | content | content |
| 0.5eq | 2.08% | 75.67% | ND | 13.92% |
| 1.0eq | 2.14% | 83.18% | ND | 11.97% |
| 1.5eq | 3.31% | 71.52% | ND | 9.00% |

* Refers to the equivalents of triethylamine used relative to compound (a-1) (eq)

TABLE 5
Experimental results of different methanol amounts

| | Remaining | Compound | Impurity | Impurity |
|---|---|---|---|---|
| Dosage of | amount of | (I-1) | B | F |
| methanol * | compound (a-1) | Content | content | content |
| 3.0eq | 1.17% | 82.29% | ND | 10.77% |
| 3.5eq | 1.80% | 78.65% | ND | 8.68% |
| 4.0eq | 2.13% | 80.57% | ND | 9.56% |
| 4.5eq | 3.17% | 86.42% | ND | 7.46% |

* Refers to the equivalents of methanol used relative to compound (a-1) (eq)

### Example 7

### Synthesis of Compound (I-1) Maleate

After impurity F was removed from compound (I-1) prepared in experiment number 7 of Table 1 of Example 4, ethyl acetate (0.63 kg) was added for redissolution, the solution was filtered through a 0.45 μm filter membrane, washed with ethyl acetate (0.4 kg), and the combined filtrates were placed into a 5 L reactor. The temperature was controlled at 20˜25° C., ethyl acetate (2 kg) solution of maleic acid (80 g) was added dropwise, after completion of addition, the temperature was maintained at 20˜25° C., and the reaction mixture was stirred for 2˜3 hours. The reaction mixture was filtered under vacuum and the filter cake was washed with ethyl acetate (0.8 kg) and dried to give 207.28 g of the white solid compound (I-1) maleate with a purity of 99.37%, an impurity B content of 0.12% and an impurity F content of 0.15%. The two-step yield of reduction (Example 4)—salt formation is 71.35%. HRMS m/z: 405.1779, 407.1736 [m+1]+1. H NMR (600 MHz, DMSO-d6) δ8.57-8.60 (q, 1H), 8.50-8.70 (brs, 1H), 7.74-7.77 (td, J=3.0 Hz, 1H), 7.61-7.63 (q, J=4.2 Hz, 1H), 7.49 (m, 1H), 7.46-7.47 (m, 1H), 7.44 (m, 1H), 7.33, 7.34 (d, J=7.2 Hz, 1H), 6.03, 6.04 (d, J=14.2, Hz, 2H), 4.41 (s, 2H), 3.70-3.73 (m, 1H), 3.63-3.65 (m, 1H), 3.52-3.60 (m, 2H), 3.45, 3.68 (d, J=9.6 Hz, 2H), 2.77-2.82 (m, 1H), 2.38-2.40 (m, 1H), 2.28-2.32 (m, 1H), 1.97-2.02 (m, 1H), 1.84, 2.39 (d, J=13.8 Hz, 2H), 1.62-1.66 (m, 1H), 1.28-1.32 (m, 1H), 1.06˜1.11 (m, 1H).

### Example 8

### Synthesis of Compound (I-2) to Compound (I-9)

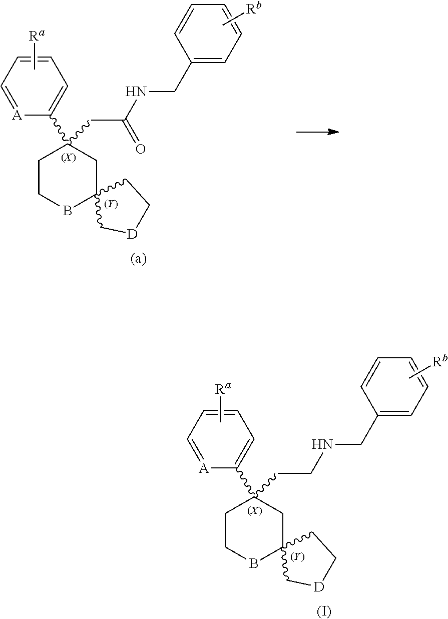

According to the above synthetic route, the maleate salts of compound (I-2) to compound (I-9) were prepared by referring to the methods of Example 4 and Example 6, and the purity of target compound was measured, respectively. The test results are shown in Table 6.

TABLE 6
Compound (I-2) to Compound (I-9) Maleate Characterization and Purity Data

| NO. | Structural Formula | ¹H NMR | MS | Purity (%) |
|---|---|---|---|---|
| (I-2) maleate salt | 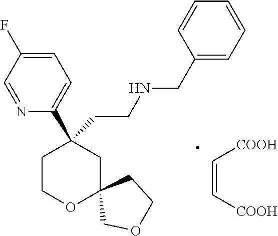 [SMILES: Fc1ccc([C@]2(CCNCc3ccccc3)CCO[C@]3(CCOC3)C2)nc1 ; O=C(O)/C=C\C(=O)O] | DMSO-d6, δ 8.49-8.59(brs, 1H), 8.53(d, J = 2.8 Hz, 1H), 7.71(m, 1H), 7.58(q, 1H), 7.30- 7.39(m, 5H), 6.00(s, 2H), 4.00(s, 2H), 3.74 (d, J = 9.2 Hz, 1H), 3.47-3.70(m, 4H), 3.41(d, J = 9.2 Hz, 1H), 2.72- 2.79(m, 1H), 2.23-2.37(m, 3H), 1.92- 2.00(m, 1H), 1.80(d, J = 14.0 Hz, 1H), 1.74- 1.84(m, 1H), 1.56-1.63(m, 1H), 1.23- 1.28(m, 1H), 1.00-1.08(m, 1H). | (I-2) [M + H]⁺ 371.2161 | 99.3% |
| (I-3) maleate salt | 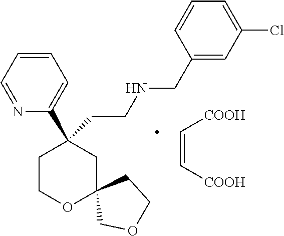 | DMSO-d6, δ 8.43-8.80(brs, 2H), 8.59(m, 1H), 7.80-7.82 (m, 1H), 7.42- 7.53(m, 4H), 7.32-7.33(m, 1H), 7.28-7.30 (m, 1H), 6.02(s, 1H), 4.04(s, 2H), 3.63- 3.74(m, 3H), 3.52-3.56(m, 2H), 3.45(d, J = 9.6 Hz, 2H), 2.78-2.82(m, 1H), 2.40-2.44(m, 2H), 2.28-2.31(m, 1H), 2.00- 2.04(m, 1H), 1.79-1.84(m, 2H), 1.62- 1.65(m, 1H), 1.27-1.28(m, 1H), 1.05- 1.10(m, 1H) | (I-3) [M + H]⁺ 387.1851 | 98.7% |
| (I-4) maleate salt | 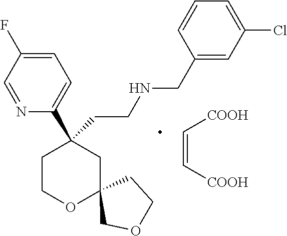 | CD3OD, δ 8.48(s, 1H), 7.55-7.60(m, 2H), 7.41-7.44(m, 3H), 7.30-7.31(m, 1H), 6.26 (s, 2H), 4.07(s, 2H), 3.73-3.80(m, 4H), 3.10(d, J = 9.0 Hz, 1H), 2.92-2.95(m, 1H), 2.82(d, J = 9.6 Hz, 1H), 2.46-2.60(m, 3H), 2.08-2.12 (m, 1H), 2.01-2.03(m, 2H), 1.89- 1.93(m, 2H), 1.77-1.80(m, 1H). | (I-4) [M + H]⁺ 405.1741 | 98.4% |
| (I-5) maleate salt | 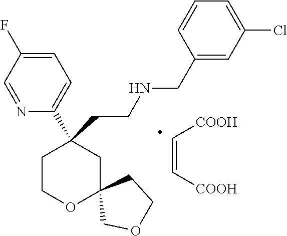 | CD3OD, δ 8.47(d, J = 3.0 Hz, 1H), 7.59- 7.62(m, 1H), 7.53-7.56(m, 1H), 7.39-7.45 (m, 3H), 7.30-7.31(m, 1H), 6.25(s, 2H), 4.07(s, 2H), 3.77-3.82(m, 2H), 3.71-3.75 (m, 2H), 3.10(d, J = 10.2 Hz, 1H), 2.90- 2.95(m, 1H), 2.92(d, J = 9.0 Hz, 1H), 2.58(d, J = 12.6 Hz, 1H), 2.46-.52(m, 2H), 2.08-2.13(m, 1H), 2.01-2.03(m, 2H), 1.88-1.95 (m, 2H), 1.76-1.81(m, 1H). | (I-5) [M + H]⁺ 405.1735 | 99.2% |
| (I-6) maleate salt | 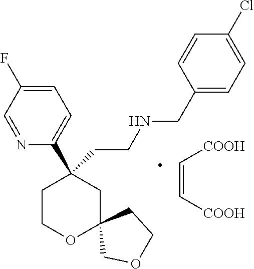 | DMSO-d6, δ 8.49-8.57(brs, 1H), 8.53(d, J = 2.8 Hz, 1H), 7.71(m, 1H), 7.57(q, J = 4.4 Hz, 1H), 7.44(d, J = 8.4 Hz, 1H), 7.41-7.47(m, 1H), 7.35 (d, J = 8.4 Hz, 1H), 7.33-7.39(m, 1H), 6.00(s, 2H), 4.00(s, 2H), 3.64(d, J = 9.2 Hz, 1H), 3.64 (d, J = 9.2 Hz, 1H), 3.45-3.70(m, 3H), 3.41(d, J = 9.2 Hz, 1H), 2.70-2.78(m, 1H), 2.45-2.47 (m, 1H), 2.21-2.36(m, 3H), 1.91-1.99(m, 1H), 1.72-1.81(m, 2H), 1.56-1.63(m, 1H), 1.23-1.28(m, 1H), 0.99-1.07(m, 1H). | (I-6) [M + H]⁺ 405.1763 | 96.1% |
| (I-7) maleate salt | 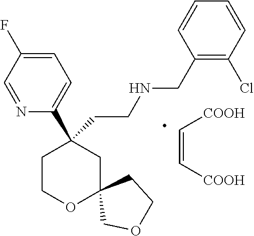 | DMSO-d6, δ 8.43-8.80(brs, 1H), 8.50-8.55 (m, 1H), 7.70-7.75(m, 1H), 7.57-7.60(m, 1H), 7.46-7.50(m, 2H), 7.35-7.40(m, 2H), 6.00 (s, 1H), 5.96-6.01(m, 1H), 4.12(s, 2H), 3.39-3.72 (m, 6H), 2.81-2.88(m, 1H), 2.28-2.40 (m, 3H), 1.93-2.03(m, 1H), 1.80-1.85(m, 2H), 1.57- 1.65(m, 1H), 1.21-1.29(m, 1H), 0.99- 1.09(m, 1H). | (I-7) [M + H]⁺ 405.1772 | 97.1% |
| (I-8) maleate salt | 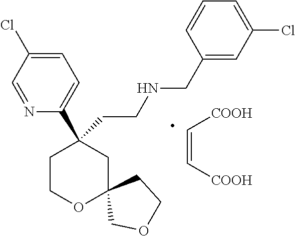 | DMSO-d6, δ 8.63(s, 1H), 7.93-7.96(m, 1H), 7.41-7.60(m, 3H), 7.34-7.36(m, 1H), 6.08(s, 2H), 3.51-3.75(m, 5H), 3.37- 3.46(m, 1H), 2.79-2.80(m, 1H), 2.30- 2.40(m, 3H), 2.00-2.01(m, 1H), 1.81- 1.87(m, 2H), 1.62-1.64 (m, 1H), 1.29- 1.34(m, 1H), 1.08~1.11(m, 1H) | (I-8) [M + H]⁺ 421.4 | 94.0% |
| (I-9) maleate salt | 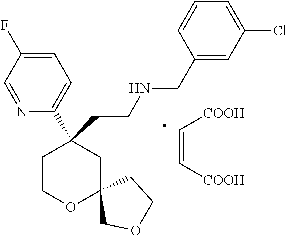 | CD3OD, δ 8.47(d, J = 2.4 Hz, 1H), 7.56- 7.62(m, 2H), 7.39-7.45(m, 3H), 7.31(m, J = 7.2 Hz, 1H), 6.28(s, 3H), 4.07(s, 2H), 3.73- 3.79(m, 3H), 3.67-3.70(m, 2H), 3.54(d, J = 9.0 Hz, 1H), 2.94-2.99(m, 1H), 2.45-2.55 (m, 3H), 2.08-2.13(m, 1H), 1.84- 1.90(m, 2H), 1.74-1.78(m, 1H), 1.12- 1.17(m, 1H), 0.86-0.91(m, 1H). | (I-9) [M + H]⁺ 405.1772 | 99.1% |

1. A method for preparing a compound of Formula (I) or a stereoisomer, a mixture of stereoisomers or salts thereof, wherein the method comprises:
reducing a compound of Formula (a) using a reducing system to afford the compound of Formula (I) according to the following scheme: 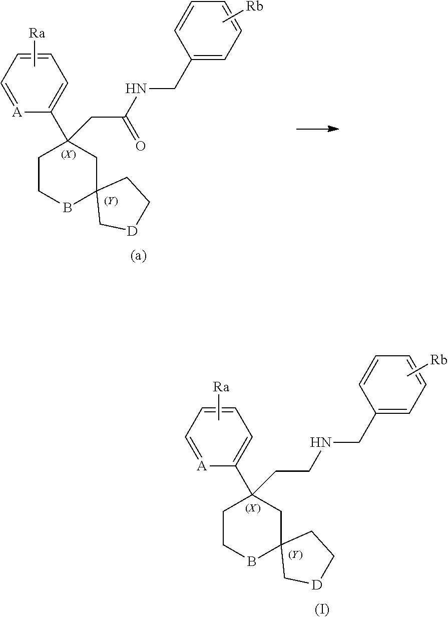
wherein: in each of the compound of Formula (I) and the compound of Formula (a),
A is C or N;
B and D are independently C, N, or O;
Ra and Rb are independently hydrogen, fluorine, chlorine, bromine, or iodine atoms; and
the carbon atoms with (X) and (Y) are chiral carbon atoms.

2. The method according to claim 1, wherein the compound of Formula (I) is selected from the group consisting of:

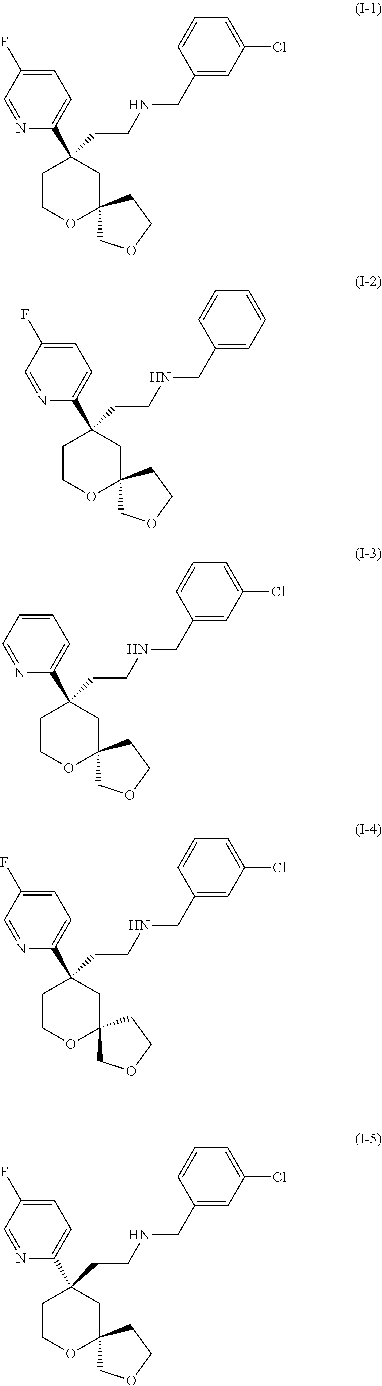 [SMILES: Fc1ccc([C@]2(CCNCc3cccc(Cl)c3)CCO[C@]3(CCOC3)C2)nc1 ; Fc1ccc([C@]2(CCNCc3ccccc3)CCO[C@]3(CCOC3)C2)nc1 ; Clc1cccc(CNCC[C@@]2(c3ccccn3)CCO[C@]3(CCOC3)C2)c1 ; Fc1ccc([C@]2(CCNCc3cccc(Cl)c3)CCO[C@@]3(CCOC3)C2)nc1 ; Fc1ccc([C@@]2(CCNCc3cccc(Cl)c3)CCO[C@]3(CCOC3)C2)nc1]

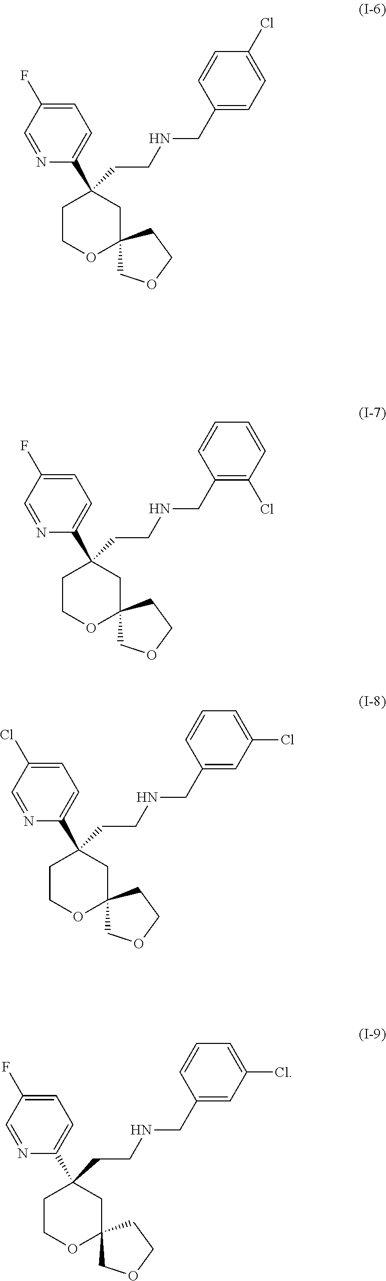 [SMILES: Fc1ccc([C@]2(CCNCc3ccc(Cl)cc3)CCO[C@]3(CCOC3)C2)nc1 ; Fc1ccc([C@]2(CCNCc3ccccc3Cl)CCO[C@]3(CCOC3)C2)nc1 ; Clc1ccc([C@]2(CCNCc3cccc(Cl)c3)CCO[C@]3(CCOC3)C2)nc1 ; Fc1ccc([C@@]2(CCNCc3cccc(Cl)c3)CCO[C@@]3(CCOC3)C2)nc1]

3. The method according to claim 1, wherein the reducing system is a mixture of NaBH₄, ZnCl₂, triethylamine, and a protic solvent.

4. The method according to claim 3, wherein the protic solvent is methanol, ethanol, isopropanol, n-butanol, formic acid, or acetic acid.

5. The method according to claim 3, further comprising preparing the compound of Formula (a) or a stereoisomer, a mixture of stereoisomers or salts thereof from a compound of Formula (b) and a compound of Formula (d) according to the following scheme: 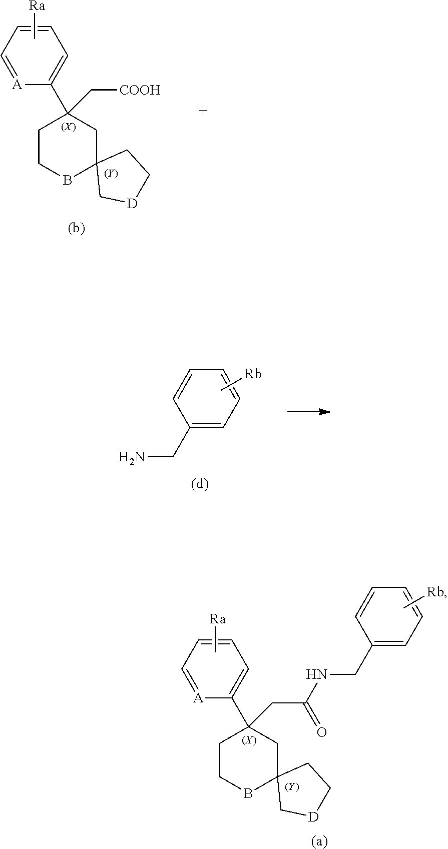
wherein:
A is C or N;
B and D are independently C, N, or O;
Ra and Rb are independently hydrogen, fluorine, chlorine, bromine, or iodine atoms; and
the carbon atoms with (X) and (Y) are chiral carbon atoms.

6. The method according to claim 5, wherein the compound of Formula (a) is prepared using a stereoisomer of the compound of Formula (b), wherein the stereoisomer of the compound of Formula (b) is a compound of Formula (b-X), wherein the compound of Formula (b-X) is prepared from the compound of Formula (b) using a chemical resolution reagent according to the following scheme:

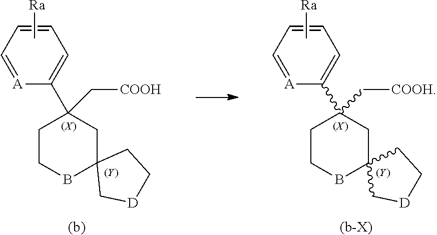

7. The method according to claim 5, wherein preparing the compound of Formula (b) or a stereoisomer, mixture of stereoisomers or salts thereof comprises:
hydrolyzing a compound of Formula (c) with a hydrolyzing agent to afford the compound of Formula (b) 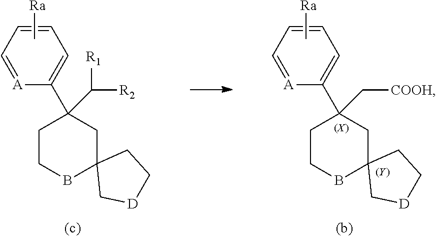
wherein:
R₁ is a cyanyl, amide, ester, carboxyl, or acyl halide group;
R₂ is a hydrogen atom, cyanyl, amide, ester, carboxyl, or acyl halide group;
A is C or N;
B and D are independently C, N, or O;
Ra is hydrogen, fluorine, chlorine, bromine, or iodine atom; and
the carbon atoms with (X) and (Y) are chiral carbon atoms.

8. The method according to claim 7, wherein the compound of Formula (b) is a racemic compound or an enantiomerically enriched form with different (X) and (Y) configurations.

9. The method according to claim 7, wherein the hydrolyzing agent comprises an acid or base, wherein the acid is sulfuric acid, methanesulfonic acid, p-toluenesulfonic acid, camphor sulfonic acid, hydrochloric acid, oxalic acid, or trifluoroacetic acid, and the base is sodium hydroxide or potassium hydroxide.

10. The method according to claim 9, wherein the compound of Formula (b) comprises a compound of Formula (b-A) and a compound of Formula (b-B), wherein a total amount of the compound of Formula (b-A) and the compound of Formula (b-B) in the compound of Formula (b) is about 95% or more by weight,

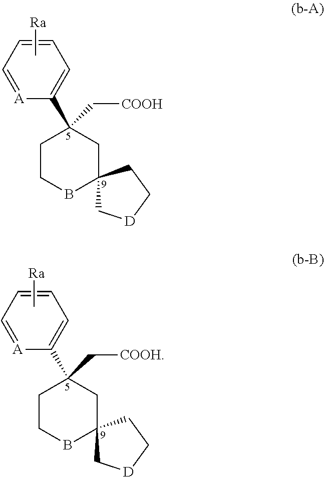

11. A mixture comprising a compound of Formula (I-1) or a salt thereof and an impurity B in an amount of less than about 0.5% by weight,
wherein the compound of Formula (I-1) is 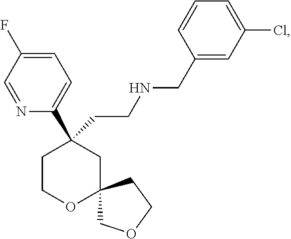 [SMILES: Fc1ccc([C@]2(CCNCc3cccc(Cl)c3)CCO[C@]3(CCOC3)C2)nc1]
and
wherein the impurity B is

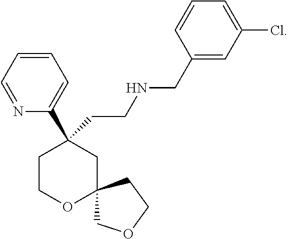 [SMILES: Clc1cccc(CNCC[C@@]2(c3ccccn3)CCO[C@]3(CCOC3)C2)c1]

12. The mixture according to claim 11, further comprising an impurity F in an amount of less than about 0.5% by weight, wherein the impurity F is

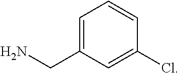

13. A compound, wherein the compound is a compound of Formula (a) or a compound of Formula (b), or a stereoisomer, a mixture of stereoisomers or salts of the compound of Formula (a) or the compound of formula (b): 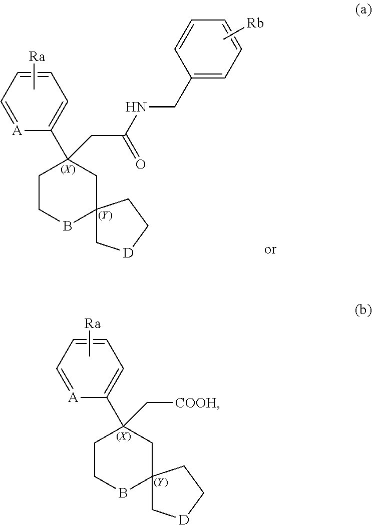
wherein: in each of the compound of Formula (a) and the compound of Formula (b),
A is C or N;
B and D are independently C, N, or O;
Ra and Rb are independently hydrogen, fluorine, chlorine, bromine, or iodine atoms; and
the carbon atoms with (X) and (Y) are chiral carbon atoms.

14. The compound according to claim 13, wherein the compound of Formula (a) is selected from the consisting of:

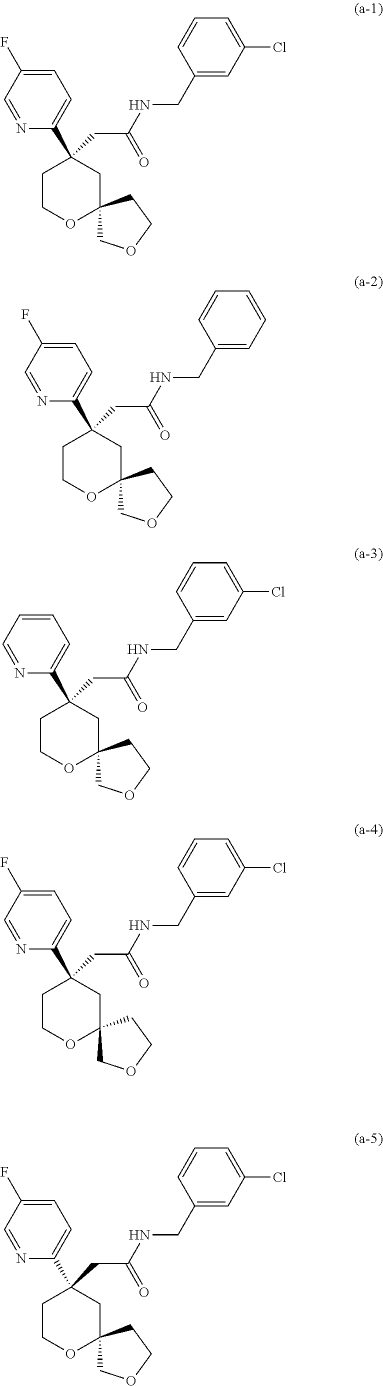 [SMILES: O=C(C[C@@]1(c2ccc(F)cn2)CCO[C@]2(CCOC2)C1)NCc1cccc(Cl)c1 ; O=C(C[C@@]1(c2ccc(F)cn2)CCO[C@]2(CCOC2)C1)NCc1ccccc1 ; O=C(C[C@@]1(c2ccccn2)CCO[C@]2(CCOC2)C1)NCc1cccc(Cl)c1 ; O=C(C[C@@]1(c2ccc(F)cn2)CCO[C@@]2(CCOC2)C1)NCc1cccc(Cl)c1 ; O=C(C[C@]1(c2ccc(F)cn2)CCO[C@]2(CCOC2)C1)NCc1cccc(Cl)c1]

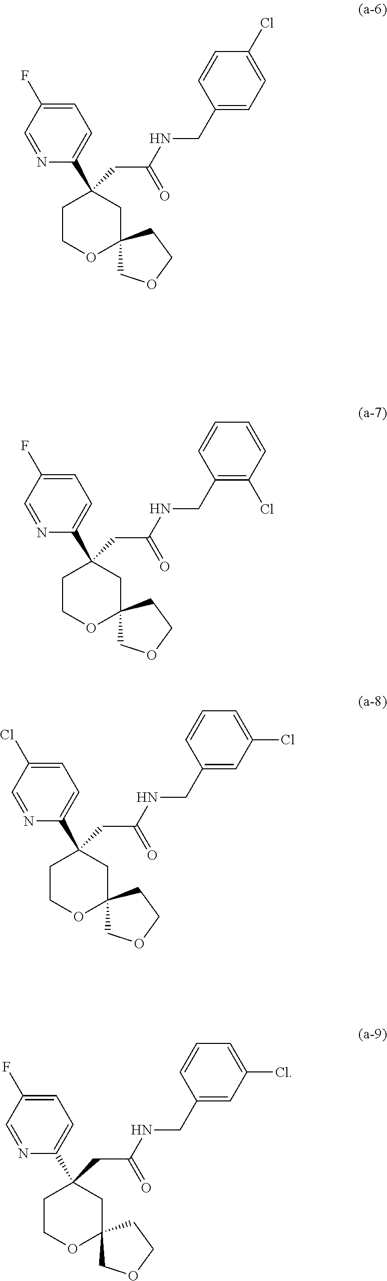 [SMILES: O=C(C[C@@]1(c2ccc(F)cn2)CCO[C@]2(CCOC2)C1)NCc1ccc(Cl)cc1 ; O=C(C[C@@]1(c2ccc(F)cn2)CCO[C@]2(CCOC2)C1)NCc1ccccc1Cl ; O=C(C[C@@]1(c2ccc(Cl)cn2)CCO[C@]2(CCOC2)C1)NCc1cccc(Cl)c1 ; O=C(C[C@]1(c2ccc(F)cn2)CCO[C@@]2(CCOC2)C1)NCc1cccc(Cl)c1]

15. The compound according to claim 13, wherein the compound (b) of Formula (b) is selected from the group consisting of: 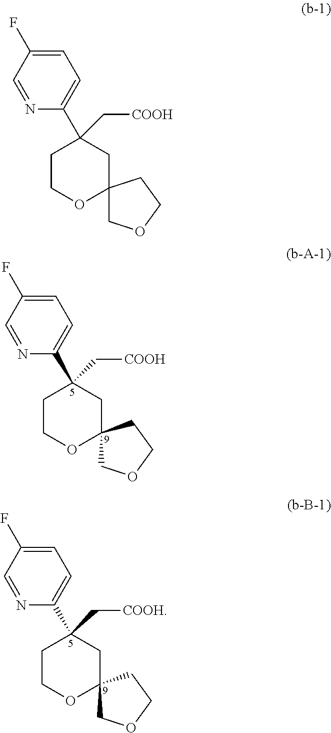 [SMILES: O=C(O)CC1(c2ccc(F)cn2)CCOC2(CCOC2)C1 ; O=C(O)C[C@@]1(c2ccc(F)cn2)CCO[C@]2(CCOC2)C1 ; O=C(O)C[C@]1(c2ccc(F)cn2)CCO[C@@]2(CCOC2)C1]
